(19) United States
(12) Patent Application Publication
(10) Pub. No.: US 2022/0170909 A1
(43) Pub. Date: Jun. 2, 2022

(54) RADIOMIC SIGNATURE FOR PREDICTING LUNG CANCER IMMUNOTHERAPY RESPONSE

(71) Applicant: H. Lee Moffitt Cancer Center and Research Institute, Inc., Tampa, FL (US)

(72) Inventors: Matthew Brian SCHABATH, Tampa, FL (US); Ilke TUNALI, Indianapolis, IN (US); Robert GILLIES, Tampa, FL (US)

(21) Appl. No.: 17/597,969

(22) PCT Filed: Aug. 15, 2020
(86) PCT No.: PCT/US2020/046570

§ 371 Date: Jan. 31, 2022

Related U.S. Application Data
(60) Provisional application No. 62/887,353, filed on Aug. 15, 2019.

(51) Int. Cl. G01N 33/50; G06T 7/45; G06T 7/00; G01N 33/68

(52) U.S. Cl. CPC G01N 33/5017; G06T 7/45; G06T 7/0012; G06T 2207/20076; G06T 2207/10081; G06T 2207/30061; G06T 2207/30096; G01N 33/6827

(57) ABSTRACT

Pre-treatment clinical data and radiomic features extracted from computed tomography (CT) scans were used to develop a parsimonious model to predict survival outcomes among NSCLC patients treated with immunotherapy. The biological underpinnings of the radiomics features were assessed utilizing geneexpression information from a well-annotated radiogenomics NSCLC dataset and were further assessed for survival in four independent NSCLC cohorts. Therefore, disclosed herein is a method for predicting efficacy of immunotherapy in a subject with lung cancer using the disclosed radiomic features.

### CROSS-REFERENCE TO RELATED APPLICATIONS

This application claims benefit of U.S. Provisional Application No. 62/887,353, filed Aug. 15, 2019, which is hereby incorporated herein by reference in its entirety.

### BACKGROUND

Immunotherapy, which targets the programmed cell death protein-1 (PD-1) or programmed cell death ligand-1 (PD-L1), has demonstrated durable clinical benefit in 20-50% patients with advanced stage non-small-cell lung cancer (NSCLC) (Reck, M. et al. N Engl J Med 375:1823-1833 (2016); Brahmer, J. et al. N Engl J Med 373:123-135 (2015); Borghaei, H. et al. N Engl J Med 373:1627-1639 (2015); Herbst, R. S. et al. Lancet 387:1540-1550 (2016); Rittmeyer, A. et al. Lancet 389:255-265 (2017); Gandhi, L. et al. N Engl J Med 378:2078-2092 (2018)). The patterns of immunotherapy response and progression are complex (Borcoman, E., et al. Am Soc Clin Oncol Educ Book 169-178 (2018)), including, e.g. rapid disease progression (Tunali, I. et al. Lung Cancer 129:75-79 (2019)), hyperprogression (Champiat, S. et al. Clin Cancer Res 23:1920-1928 (2017)), and acquired resistance (Sharma, P., et al. Cell 168:707-723 (2017)). Because of this complexity, there is a pressing challenge to identify robust predictive biomarkers that can identify patients that are least likely to respond. Though tumor PD-L1 expression by immunohistochemistry (IHC) is the only clinically approved biomarker to predict immunotherapy response, recent clinical trials demonstrated significant improvements in clinical outcomes irrespective of PD-L1 expression level (Gandhi, L. et al. N Engl J Med 378:2078-2092 (2018); Antonia, S. J. et al. N Engl J Med 377:1919-1929 (2017)). Furthermore, tumor mutational burden (TMB), defined as the total number of mutations per coding area of a tumor genome (Yarchoan, M., et al. N Engl J Med 377:2500-2501 (2017)), has been shown to be a superior predictor of immunotherapy response compared to PD-L1 status (Hellmann, M. D. et al. Cancer Cell 33:843-852 e844 (2018); Hellmann, M. D. et al N Engl J Med 378:2093-2104 (2018); Cristescu, R. et al. Science 362 (2018)). Despite the potential clinical utility of TMB, there are limitations with its use as tumor specimens have to be sufficient in both quantity and quality (Hellmann, M. D. et al. N Engl J Med 378:2093-2104 (2018)). Further, tumors are evolutionarily dynamic and accumulate mutations rapidly (Goodman, A. M. et al. Mol Cancer Ther 16:2598-2608 (2017)), and laboratory methods to calculate TMB can be timely and expensive. Moreover, tumor-based biomarkers, including PD-L1 expression, are often subject to sampling bias due to the molecular and cellular heterogeneity of the biopsied tumors (Gerlinger, M. et al. N Engl J Med 366:883-892 (2012)). As such, complimentary biomarkers that are predictive, non-invasive, and measured in a timely fashion would have direct translational implications.

### SUMMARY

Pre-treatment clinical data and radiomic features extracted from computed tomography (CT) scans were used to develop a parsimonious model to predict survival outcomes among NSCLC patients treated with immunotherapy. The biological underpinnings of the radiomics features were assessed utilizing gene-expression information from a well-annotated radiogenomics NSCLC dataset and were further assessed for survival in four independent NSCLC cohorts.

Therefore, disclosed herein is a method for predicting efficacy of immunotherapy in a subject with lung cancer that involves receiving image data from contrast-enhanced thoracic computed tomography (CT) scans, and using a data processor to process the image texture data utilizing gray level co-occurrence matrix (GLCM) inverse difference feature. The GLCM inverse difference is an “avatar feature” that is correlated with nine other radiomic features (FIG. 2B). In some embodiments, high GLCM inverse difference indicates reduced efficacy of the immunotherapy in the subject.

GLCM inverse difference can be used alone or in combination with other predictive factors, such as the number of metastatic sites and blood serum albumin levels. In some embodiments, the method further involves determining the number of metastatic sites in the subject, wherein elevated metastatic sites indicates reduced efficacy of the immunotherapy in the subject. In some embodiments, the method further involves assaying a blood sample from the subject for serum albumin, wherein a high GLCM inverse difference and decreased levels of serum albumin indicates reduced efficacy of the immunotherapy in the subject. In some embodiments, high GLCM inverse difference, reduced levels of serum albumin and elevated metastatic sites indicates reduced efficacy of the immunotherapy in the subject.

Also disclosed is a method for treating a subject with lung cancer that involves receiving image data from contrast-enhanced thoracic computed tomography (CT) scans, using a data processor to process the image data and detect low gray level co-occurrence matrix (GLCM) inverse difference feature indicative of less dense and less uniform lesions, and treating the subject with immunotherapy.

In some embodiments, the method further involves detecting no more than 1 metastatic sites in the subject, indicating that the subject is more likely to respond to immunotherapy. In some embodiments, the method further involves detecting at least 3.9 g/dL serum albumin in a blood sample from the subject, indicating that the subject is more likely to respond to immunotherapy. In some embodiments, a low GLCM inverse difference and reduced number of metastatic sites indicates improved efficacy of the immunotherapy in the subject. In some embodiments, the metastatic site range is 1 to 6. In some embodiments, the GLCM inverse difference range is 0.27 to 0.80.

The disclosed immunotherapy can be a checkpoint inhibitor. The two known inhibitory checkpoint pathways involve signaling through the cytotoxic T-lymphocyte antigen-4 (CTLA-4), programmed-death 1 (PD-1) receptors, and programmed death ligand-1 (PD-L1). These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHlgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MED14736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows an example radiomics pipeline disclosed herein. Baseline, pre-treatment patient data is obtained, including: clinical covariates and computational image-based features (Radiomics). Radiomic features are extracted from standard-of-care imaging studies (yellow). Radiologists mark the target lesions, the lesions are automatically segmented, and radiomic features are extracted from the ROI (purple). Unstable, non-reproducible and correlated radiomic features are removed. The remaining features are combined with the clinical covariates (green) and predictive model building approaches are applied which can be used for patient stratification and treatment selection.

FIG. 2A is a heat map of concordance correlation coefficients (CCC) for different segmentations and image acquisitions of radiomic features. Each column in the heat map represents a radiomic feature from the indicated feature group and region-of-interest (e.g., intratumoral or peritumoral). The features are compared between different segmentation algorithms (ALG), different initial parameters (IP) and between test-retest scans (RIDER dataset). The green boxes represent higher (CCC>0.95), blue boxes represent moderate (CCC≥0.75 & CCC≤0.95) and red boxes represent lower (CCC<0.75) CCCs. FIG. 2B is a correlation matrix for the radiomic features that were significantly associated with overall survival in the univariable analysis. The feature in the final parsimonious model was GLCM inverse difference (also referred to as “average co-occurrence inverse difference”) and it is found to be correlated with nine other features shown inside the green box. FIG. 2C shows the Classification and Regression Tree (CART) was used to identify patient risk groups based on a model containing one radiomic feature and two clinical features. Patients were grouped from low risk to very high risk based on the CART decision nodes and terminal nodes.

FIGS. 3A to 3D are Kaplan-Meier survival curves estimates for overall survival in the training (FIG. 3A) and test cohorts (FIG. 3B), and progressive-free survival in the training (FIG. 3C) and test cohorts (FIG. 3D).

FIGS. 4A and 4B show overall survival (FIG. 4A) and progression-free survival (FIG. 4B) for the training and test cohorts.

FIGS. 5A and 5B show overall survival (FIG. 5A) and progression-free survival (FIG. 5B) for the six risk groups identified by CART in the training cohort. Groups 2 and 3 and groups 4 and 5 were combined for the analyses in FIG. 3.

FIGS. 6A and 6B shows time-dependent AUC curves for Cox regression models based on 6, 12, 24 and 36 months for training (FIG. 6A) and test cohorts (FIG. 6B). The AUC values were statistically not different between training and test cohorts.

FIGS. 7A and 7B are whisker-box plots of CAIX expression from the radio-genomics dataset comparing low (≤0.43) vs. high (>0.43)-GLCM inverse difference.

FIGS. 8A to 8F are Kaplan-Meier survival plots of patients dichotomized by radiomics score. Same cut-off point was used for dichotomizing the training cohort (FIG. 8A), test cohort (FIG. 8B), Gene-expression cohort (FIG. 8C), NLST cohort (FIG. 8D), Moffitt adenocarcinoma cohort (FIG. 8E), and MAASTRO adenocarcinoma cohort (FIG. 8F).

FIG. 9 are CT scans of patients in low and very-high risk groups. First column represents the primary target lesion CT scan. Second column represents the tumor segmentation. Third column represents a gradient image of the segmented area for visualization of the tumor texture. Patient on the top was identified as a low risk patient to immunotherapy and had a less dense tumor phenotype with lower GLCM inverse difference score. Patient on the bottom was identified as a very-high risk patient and had a dense tumor phenotype with higher GLCM inverse score.

FIGS. 10A and 10B are Kaplan-Meier survival plots of patients dichotomized by metastatic site number. Same cut-off point was used for dichotomizing the training cohort (FIG. 10A) and test cohort (FIG. 10B).

FIGS. 11A and 11B are Kaplan-Meier survival plots of patients dichotomized by serum albumin. Same cut-off point (3.9) was used for dichotomizing the training cohort (FIG. 11A) and test cohort (FIG. 11B).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

Grey Level Co-Occurrence Matrix Based Features

The grey level co-occurrence matrix (GLCM) has been proved to be a powerful approach for image texture analysis. The grey level co-occurrence matrix (GLCM) is a matrix that expresses how combinations of discretized grey levels of neighboring pixels (or voxels in 3 dimensional space) in a region-of-interest are distributed along one of the spatial image directions. In other words, it describes how often a pixel of grey level i appears in a specific spatial relationship to a pixel of grey level j. Hence GLCM matrix defined as P where each matrix element pᵢⱼ=P(i, j) represents the number of times a grey level i is neighbors with voxels of grey level j with an inter-pixel distance and orientation. The GLCM defines a square matrix whose size is equal to the largest grey level N_{g} appearing in the region-of-interest. Haralick et al (Haralick et al. IEEE Trans Syst Man Cybern, 6:610-621,1973) proposed 14 original statistics (e.g., contrast, correlation, energy) to be applied to the GLCM to measure the texture features. The GLCM inverse difference feature (i.e., “avatar” feature) is a measure of homogeneity, where the feature quantity is greatest if all grey levels are the same. Inverse difference is defined as follows:

$${F}_{glcm . inv . diff} = {\sum }_{i = 1}^{{N}_{g}} {\sum }_{j = 1}^{{N}_{g}} \frac{{p}_{ij}}{1 + \vert i - j \vert}$$

where N_{g} is the number of discretized grey levels inside the region-of-interest.

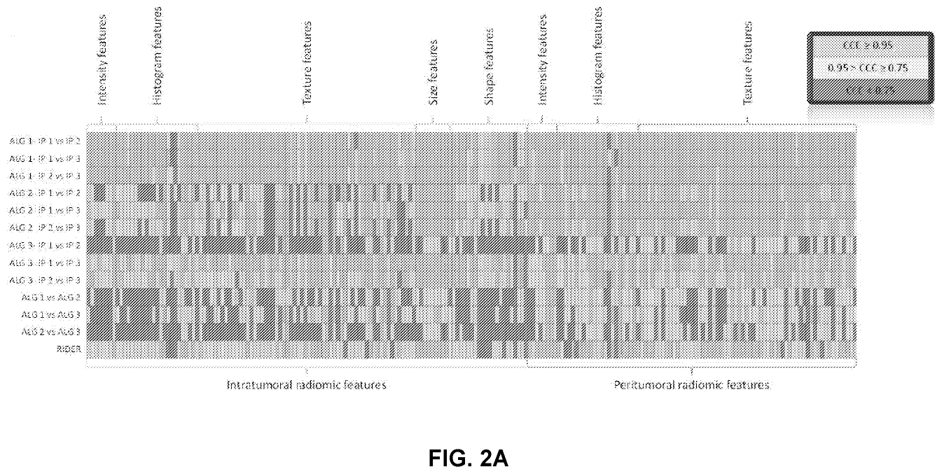

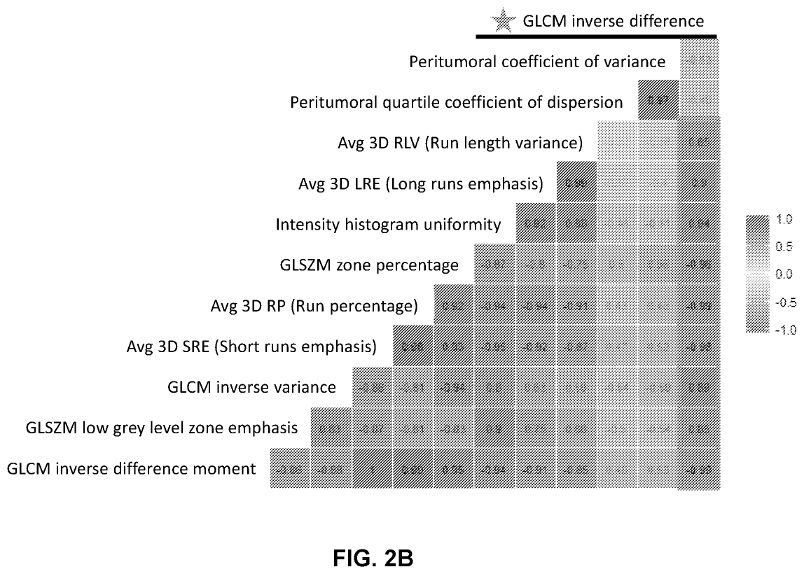

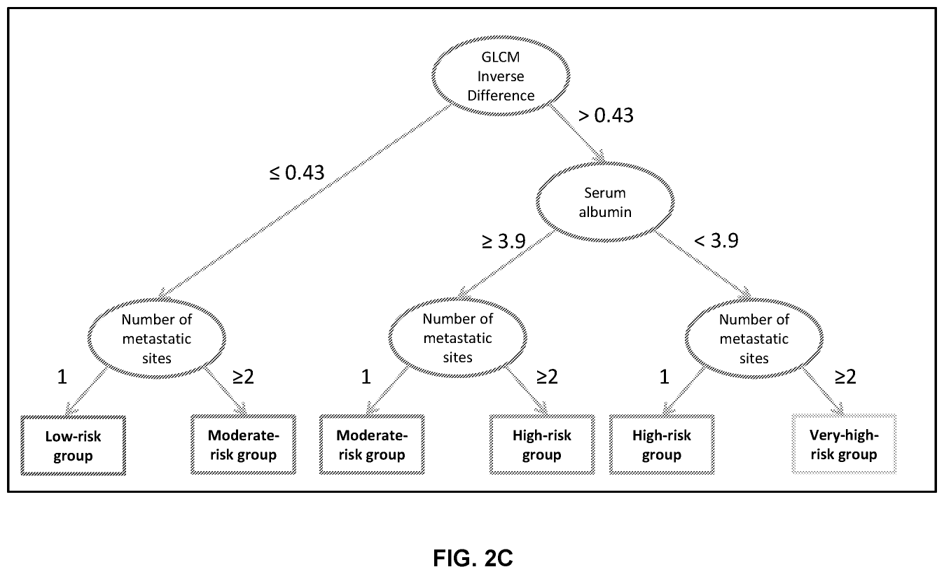

The features that were found to be correlated with GLCM inverse difference were presented in FIG. 2B. Two other features were calculated using GLCM as follows:
i) GLCM Inverse Difference Moment:

$${GLCM}_{inverse difference moment} = {\sum }_{i = 1}^{{N}_{g}} {\sum }_{j = 1}^{{N}_{g}} \frac{{p}_{ij}}{1 + {( i - j )}^{2}}$$

ii) GLCM inverse variance:

$${GLCM}_{inverse variance } = 2 {\sum }_{i = 1}^{{N}_{g}} {\sum }_{j > 1}^{{N}_{g}} \frac{{p}_{ij}}{{( i - j )}^{2}}$$

Grey Level Run Length Based Features

The grey level run length matrix (GLRLM) features were first presented by Galloway (M. Galloway, Computer Graphics and Image Processing, 4:172-179, 1975) to quantify texture of an image or region-of-interest. GLRLM is a matrix that consists of counts of the grey level run lengths along a desired spatial direction. A GLRLM is defined as R where each matrix element R(i, j) represents the number of runs of a grey level i of length j. Hence the GLRLM is sized N_{g}×Nᵣ. Four features utilizing GLRLM were found to be correlated with GLCM inverse difference (FIG. 2B).

Avg 3D RLV (Run Length Variance):

$${GLRLM}_{rlv } = {\sum }_{i = 1}^{{N}_{g}} {\sum }_{j = 1}^{{N}_{g}} {( j - µ )}^{2} {p}_{ij}$$

Where pᵢⱼ=rᵢⱼ/Nₛ and the mean run length μ=Σᵢ₌₁^{N}_{g} Σⱼ₌₁^{N}ᵣjPᵢⱼ
i) Avg 3D LRE (Long runs emphasis):

$$G L R L {M}_{lre} = \frac{1}{{N}_{s}} {\sum }_{j = 1}^{{N}_{r}} {j}^{2} {r}_{j}$$

where rⱼ=Σᵢ₌₁^{N}_{g} rᵢⱼ.
ii) Avg 3D RP (Run percentage):

$$G L R L {M}_{rp} = \frac{{N}_{s}}{{N}_{v}}$$

Where Nᵥ is the total number of voxels in a region-of-interest (ROI).
iii) Avg 3D SRE (Short runs emphasis):

$$G L R L {M}_{sre} = \frac{1}{{N}_{s}} {\sum }_{j = 1}^{{N}_{r}} \frac{{r}_{j}}{{j}^{2}}$$

Grey Level Size Zone Based Features

The grey level size zone matrix (GLSZM) counts the number of groups (i.e., zones) of connected pixels with an explicit discretized grey level value and size and was first proposed by Thibault et al (Thibault et al. 2014). The voxel connectedness depends on the desired definition of connectedness where in 3 dimensional approaches is 26-connectedness and in 2 dimensional approaches is 8-connectedness. A GLSZM defined as S is sized as N_{g}×N_{z} where N_{g} is the number of discretized grey levels in the region-of-interest and N_{z} is the maximum zone size. Each element sᵢⱼ=s(i,j) is the number of zones with discretized grey level i and size j.

One feature utilizing GLSZM were found to be correlated with GLCM inverse difference (FIG. 2B).

GLSZM Low Grey Level Zone Emphasis:

$$G L S Z {M}_{glze} = \frac{1}{{N}_{s}} {\sum }_{i = 1}^{{N}_{g}} \frac{{s}_{i}}{{i}^{2}}$$

where sⱼ=Σᵢ₌₁^{N}_{g}sᵢⱼ.

Intensity Histogram Features

An intensity histogram is a graph of pixel intensities versus number of pixels. The pixel intensities are usually discretized by the original set of grey levels into grey level bins. Assuming there are N_{y} number of discretized grey levels, the probability of occurrence of each grey level bin i is pᵢ=nᵢ/Nᵥ.

One feature utilizing intensity histogram was found to be correlated with GLCM inverse difference (FIG. 2B).

Intensity Histogram Uniformity:

$${H}_{uni} = {\sum }_{i = 1}^{{N}_{g}} {p}_{i}^{2}$$

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Clinical Factors and Quantitative Image-Based Features Predict Immunotherapy Response Among Lung Cancer Patients

Materials and Methods

Immunotherapy-Treated Lung Cancer Patients

This analysis included 270 stage IIIB or IV NSCLC patients that were treated with immunotherapy using PD-1 single agent (Nivolumab, Pembrolizumab), PD-L1 single agent (Durvalumab, Atezolizumab), or combination of PD-L1 or PD-L1 with cytotoxic T-lymphocyte-associated protein 4 (Ipilimumab, Tremelimumab) as second agent. Inclusion criteria included patients having a baseline CT or PET/CT scan less than 90 days prior to the initiation of immunotherapy and at least one Response Evaluation Criteria in Solid Tumors (RECIST) target or non-target lung lesion. The patients were divided into training (N=180) and test cohorts (N=90). Patients in the training cohort were enrolled in clinical trials treated between Jun. 2011 and Jan. 2016 at Moffitt Cancer Center. Details of these patients have been previously published⁸. Patients in the test cohort were treated with immunotherapy between May 2015 and Oct. 2017 where 94.6% were treated as standard-of-care and 5.4% were enrolled in industry-sponsored clinical trials at Moffitt Cancer Center. Patient data were obtained from electronic medical records and institutional databases including demographics, stage of disease, histology, treatment, vital status, targeted mutations, ECOG performance, RECIST, and hematology data. Moffitt's Cancer Registry collects vital status (deceased or alive) through active (i.e., chart reviews and directly contacting the patients, relatives, and other medical providers) and passive methods (i.e., mortality records). Progression was abstracted and defined as progressive disease from RECIST definition or clinical progression evaluated by the treating clinicians whenever RECIST was not available.

Assessing Stable and Reproducible Features

Two separate publicly available datasets were utilized to assess stability (The Moist-run dataset 1), and reproducibility (RIDER dataset 2) of radiomic features to increase the likelihood of a reproducible and robust radiomics model.

The Moist-run dataset was constructed by the Quantitative Imaging Network (QIN) as part of a lung segmentation challenge (Kalpathy-Cramer, J. et al. J Digit Imaging 29:476-487 (2016)) and consists of 40 chest CT images of 40 NSCLC patients and one thoracic phantom from five collections of Digital Imaging and Communications Medicine series. Each patient in the dataset had one lesion of interest and the thoracic phantom scan had 12 lesions of interest. The RIDER test-retest dataset which was used to find the reproducible features (Zhao, B. et al. Radiology 252:263-272 (2009)) consisted of 32 NSCLC patients with two separate non-contrast CT scans acquired within 15 minutes of each other using the same scanner with fixed acquisition and processing parameters. As such, the only variation between the test and retest scans were attributed to patient orientation, respiratory, and movement. The images on these datasets were previously de-identified

Using the Moist run dataset, all radiomic features were computed for 9 different segmentations done by 3 different algorithms which each were run by 3 different initial parameters. Afterwards, concordance correlation coefficient (CCC) metric was calculated to assess inter- and intra-segmentation differences of the radiomic features. The RIDER dataset was utilized to assess reproducibility of radiomic features between test and re-test scans. After extracting radiomic features from both scans of the patients, CCC values were calculated and features that have a CCC<0.75 were eliminated.

Shape features were only extracted from intratumoral regions as they were proven to be highly correlated (Pearson correlation>0.95) with their peritumoral versions.

Radiogenomics Dataset

A previously described dataset (Schabath, M. B. et al. Oncogene 35:3209-3216 (2016)) of 103 surgically resected adenocarcinoma patients who had pre-surgery CTs and gene expression data was used to identify potential biological underpinnings of the most informative radiomic feature. Briefly, gene expression was IRON-normalized and batch-corrected for RNA quality Pathway and Gene Ontology Enrichment was performed using Clarivate Analytics MetaCore.

Prognostic Validation Datasets

The radiomics data were further validated for prognosis in four published datasets. Only OS was available for these datasets. The first dataset (Grove, O. et al. PLoS One 10:e0118261 (2015); Tunali, I. et al. Oncotarget 8:96013-96026 (2017)) comprised of 62 adenocarcinoma patients who underwent surgical resection as first course therapy at the Moffitt Cancer Center and had pre-surgery CTs within 2 months prior to surgery. The second dataset (Grove, O. et al. PLoS One 10:e0118261 (2015); Tunali, I. et al. Oncotarget 8:96013-96026 (2017)) comprised of 47 adenocarcinoma patients who underwent surgical resection as first course therapy at the Maastricht Radiation Oncology Clinic and had pre-surgery CTs within 2 months prior to surgery. The third dataset included 234 patients (Hawkins, S. et al. J Thorac Oncol 11:2120-2128 (2016); Liu, Y. et al. Radiology 286:298-306 (2018)) diagnosed with screen-detected incident lung cancers in the National Lung Screening Trial. The fourth dataset was a radiogenomics dataset (Schabath, M. B. et al. Oncogene 35:3209-3216 (2016)) of 103 adenocarcinoma patients as described above.

Tumor Segmentation and Radiomics Extraction

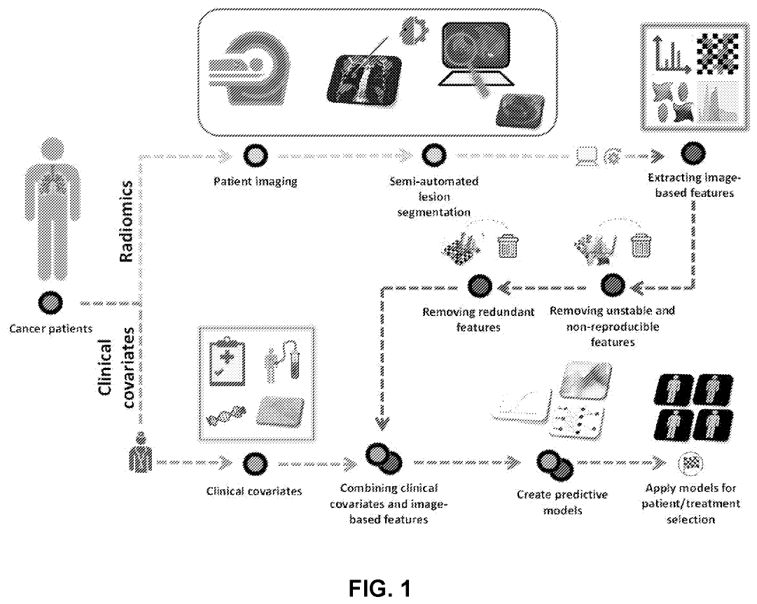

FIG. 1 presents an overview of the radiomics pipeline. Pre-treatment contrast-enhanced thoracic CT scans performed s 90 days (median: 10 days) prior to the initiation of immunotherapy (baseline) were retrieved from the picture archiving and communication system and loaded into HealthMyne Quantitative Imaging Decision Support (QIDS) software. A radiologist (Y. T.) with more than 10 years of clinical experience selected the largest lung tumor of the patients and initialized an automated 3D segmentation algorithm using the HealthMyne® QIDS Rapid Precise Metrics software. The tumor delineation outputs of the 3D segmentation algorithm were either confirmed or edited whenever necessary by the radiologist.

The tumor mask images (i.e., tumor delineations) were imported into an in-house radiomic feature extraction toolboxes created in MATLAB® 2015b (The Mathworks Inc., Natick, Mass.) and C++. The CT images were resampled to a single voxel spacing of 1 mm×1 mm×1 mm using cubic interpolation to standardize spacing across all images. Hounsfield units (HU) in all CT images were then resampled into fixed bin sizes of 25 HUs discretized from −1000 to 1000 HU.

A total of 213 radiomic features were extracted utilizing the training cohort from the intratumoral region (N=122 features) and the peritumoral region 3 mm outside of tumor boundary (N=91 features) using standardized algorithms from the Image Biomarker Standardization Initiative (IBSI) v5 (Zwanenburg, A., et al.). Peritumoral regions were bounded by the lung parenchyma mask to exclude any tissue that exceed outside of the lung parenchyma. Unstable and non-reproducible radiomic features were eliminated utilizing two publicly available datasets (Kalpathy-Cramer, J. et al. J Digit Imaging 29:476-487 (2016); Zhao, B. et al. Radiology 252:263-272 (2009)). The “Moist-run” dataset²⁵ was utilized to identify stable features which consist of 40 CT images of lung tumors with three different segmentation algorithms and three different initialization parameters (e.g., seed point) for each segmentation. The Reference Image Database to Evaluate Therapy Response (i.e., RIDER) test-retest dataset (Zhao, B. et al. Radiology 252:263-272 (2009)) was used to identify reproducible features which consists of 32 lung cancer patients who had two non-contrast chest CT scans acquired 15 minutes apart using the same scanner, acquisition, and processing parameters. Stable features were identified by assessing the concordance correlation coefficient (CCC) between radiomic features extracted using different segmentations from the “Moist-run” dataset. Reproducible features were identified by assessing the CCC between radiomic features extracted test and the retest scans from the RIDER dataset.

Statistical Analysis

All statistical analyses were performed using Stata/MP 14.2 (StataCorp LP, College Station, Tex.) and R Project for Statistical Computing version 3.4.3. Differences for the clinical covariates were tested using Fisher's exact test for categorical variables and the Mann-Whitney U test for continuous variables. Survival analyses were performed using Kaplan-Meier survival estimates and the log-rank test. The OS and progression-free survival (PFS) were the two dependent variables. For OS, an event was defined as death and the data were right censored at 36-months. For PFS, an event was defined as death or either clinical or RECIST based progression of cancer and the data were right-censored at 36 months. The index date for both OS and PFS was the date of initiation of immunotherapy.

A rigorous model building approach was employed to reduce the number of covariates and identify the most informative clinical covariates and radiomic features associated with patient survival. For the clinical covariates, univariable Cox regression was performed and covariates significantly (P<0.05) associated with OS were retained. To produce a parsimonious clinical model, the remaining clinical covariates were included in a stepwise backward elimination Cox regression model using a threshold of 0.01 for inclusion. For the radiomic features, univariable Cox regression was performed and radiomic features were retained that were significantly associated with OS after Bonferroni-Holm correction (P<0.05). Radiomic features correlated with tumor volume (Pearson's correlation coefficient 0.80) were removed. Among the remaining radiomic features, correlated features were identified using an absolute Pearson's correlation coefficient 0.80 and the feature with the smallest p-value from the univariable analysis was retained. The remaining radiomic features, were utilized to identify a parsimonious radiomics model using a stepwise backward elimination approach applying a threshold of 0.01 for inclusion. The final covariates from the clinical model and the final features from the radiomics model were combined and Classification and Regression Tree (CART) was used to find patient risk groups. CART is a non-parametric approach modified for failure time data (Breiman, L. New York, N.Y.: Kluwer Academic Publishers (1984)) that classifies variables through a decision tree composed of splits, or nodes, where the split points are optimized based on impurity criterion. The clinical-radiomics CART model from the training cohort and was validated utilizing the test cohort. Time-dependent AUCs and confidence intervals (Cl) were calculated for 6, 12, 24 and 36 months for training and test cohorts. The most predictive radiomics feature was also validated in four independent cohorts.

For the radiogenomics analysis, the highest prognostic radiomic feature was compared to every gene probesets using two different approaches: correlation and two-group analysis. For the correlation analysis, gene probesets were filtered and determined as statistically significant using the following criteria: Pearson's correlation with a threshold |R|>0.4, an expression filter with max expression of gene>5, and an inter-quartile filter (IQR>log 2 (1.2 FC)). Gene probesets were filtered and determined as significant using the following criteria based on a Student's t test p<0.001 and mean log fold-change between high and low prognostic radiomic feature oflfc>log 2 (1.4 FC). The significant probesets from the two analyses were intersected yielding a final list of probesets significantly associated with the prognostic radiomic feature.

Results

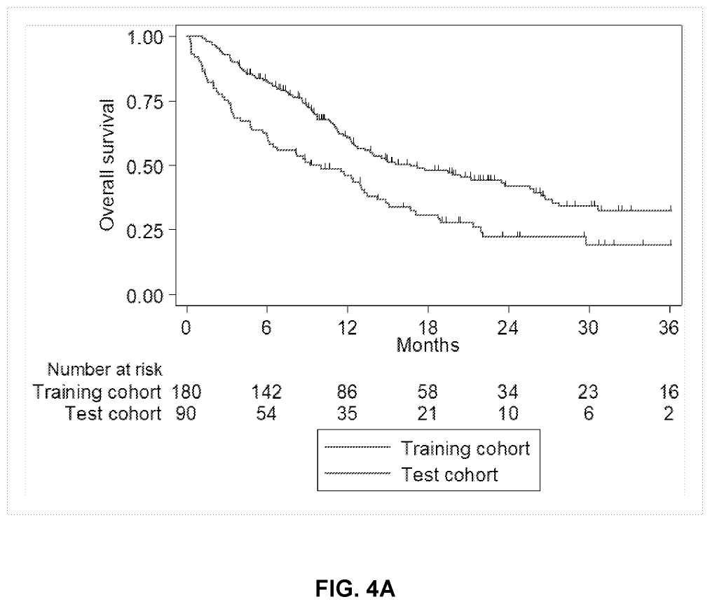

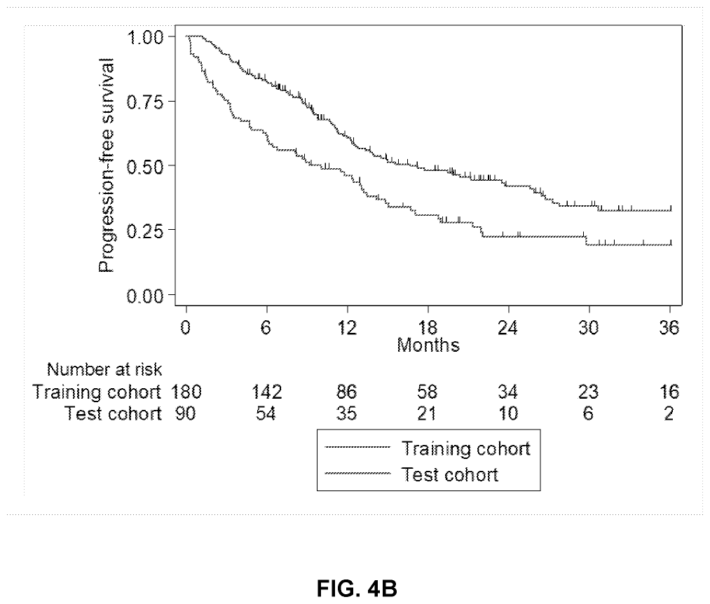

Immunotherapy treated patient demographics: Type of checkpoint inhibitor, ECOG performance status, number of previous lines of therapy, serum albumin, lymphocyte counts, and neutrophils to lymphocytes ratio (NLR) were significantly different between the training and test cohorts (Table 1). Also, significant differences were found for OS and PFS between training and test cohorts (36-month OS 32.6% vs. 19.2%, respectively; 36-month PFS 20.8% vs. 9.5%, respectively; Table 2) where log-rank P-value was <0.05 (FIG. 4).

TABLE 1
Patient characteristics by the training and test cohorts

| | | | |
|---|---|---|---|
| | Training Cohort | Test Cohort | |
| Characteristic | (N = 180) | (N = 90) | P-Value |

Age at initiation of treatment, N (%)

| | | | |
|---|---|---|---|
| Dichotomized | | | |
| <65 | 68 (37.8) | 37 (41.1) | |
| ≥65 | 112 (62.2) | 53 (58.9) | 0.599 |
| Median, (95% CI) | 67 (65-68) | 67 (64-69) | 0.783 |

Sex, N (%)

| | | | |
|---|---|---|---|
| Female | 95 (52.8) | 43 (47.8) | |
| Male | 85 (47.2) | 47 (52.2) | 0.442 |

Smoking status¹

| | | | |
|---|---|---|---|
| Never smoker | 30 (16.7) | 16 (17.8) | |
| Ever smoker | 146 (81.1) | 74 (82.2) | 0.866 |
| Unknown/Missing | 4 (2.2) | 0 (0) | |

Stage, N (%)

| | | | |
|---|---|---|---|
| IIIb | 6 (3.3) | 4 (4.4) | |
| IV | 174 (96.7) | 86 (95.6) | 0.735 |

Histology, N (%)

| | | | |
|---|---|---|---|
| Adenocarcinoma/others | 137 (76.1) | 71 (78.9) | |
| Squamous cell carcinoma | 43 (23.9) | 19 (21.1) | 0.648 |

Checkpoint inhibitors, N (%)

| | | | |
|---|---|---|---|
| Anti PD-L1 | 48 (26.6) | 18 (20.0) | |
| Anti PD-1 | 57 (31.7) | 69 (76.7) | |
| Doublet | 75 (41.7) | 3 (3.3) | <0.001 |

ECOG performance status, N (%)

| | | | |
|---|---|---|---|
| 0 | 39 (21.7) | 10 (11.1) | |
| 1 | 141 (78.3) | 67 (74.4) | |
| 2 | 0 (0) | 13 (14.4) | <0.001 |

Previous lines of therapy on current diagnosis

| | | | |
|---|---|---|---|
| None | 70 (43.9) | 21 (23.3) | |
| 1 | 48 (26.7) | 47 (52.2) | |
| ≥2 | 62 (34.4) | 22 (24.4) | <0.001 |

Number of metastatic sites

| | | | |
|---|---|---|---|
| 1 | 82 (46.6) | 51 (56.7) | |
| ≥2 | 98 (54.4) | 39 (43.3) | 0.094 |

EGFR mutational status¹

| | | | |
|---|---|---|---|
| Not Detected | 107 (59.4) | 37 (41.1) | |
| Detected | 25 (13.9) | 5 (5.6) | |
| Missing/Inconclusive | 48 (26.7) | 48 (53.3) | 0.355 |

KRAS mutational status¹

| | | | |
|---|---|---|---|
| Not Detected | 61 (33.9) | 20 (22.2) | |
| Detected | 29 (16.1) | 12 (13.3) | 0.664 |
| Missing/Inconclusive | 90 (50.0) | 58 (64.4) | |

Hematology, median, (95% CI)

| | | | |
|---|---|---|---|
| Serum albumin, (g/dL) | 4.0 (3.9-4.0) | 3.8 (3.6-3.9) | <0.001 |
| Lymphocytes, (1e + 9/L) | 1.3 (1.2-1.4) | 1.0 (0.9-1.2) | <0.001 |
| WBC, (1e + 9/L) | 7.1 (6.7-7.6) | 7.7 (6.8-8.8) | 0.246 |
| Neutrophils, (1e + 9/L) | 4.8 (4.4-5.1) | 5.3 (4.6-6.5) | 0.131 |
| Ratio of: | 3.7 (3.2-4.1) | 5.2 (4.0-7.5) | 0.002 |
| Neutrophils/Lymphocytes | | | |

Abbreviations:
CI = confidence interval;
NLR = neutrophils to lymphocytes ratio;
Bold P-values are statistically significant;
P-values for continuous variables were calculated using Mann-Whitney test and Fisher's Exact Test for categorical variables.
¹P-values for smoking status, EGFR mutational status and KRAS mutational status were calculated for patients without missing/inconclusive data.
Majority of the training (95.3%) and the test cohort (86.7%) were self-reported White race.
Majority of the training (97.0%) and the test cohort (88.9%) were self-reported non-Hispanic.

TABLE 2
Overall survival and progression free survival rates by training and test
cohorts and patient risk groups¹

| | | Percent survival at: | | | |
|---|---|---|---|---|---|
| | | 6 | 12 | 24 | 36 |
| | | months | months | months | months |
| Overall survival | | | | | |
| Overall by cohort | | | | | |
| | Training Cohort | 82.7% | 60.8% | 42.1% | 32.6% |
| | Test Cohort | 61.7% | 46.2% | 22.4% | 19.2% |
| By risk group | | | | | |
| Low-risk | | | | | |
| | Training Cohort | 100% | 95.2% | 84.7% | 84.7% |
| | Test Cohort | 95.0% | 85.0% | 38.9% | 38.9% |
| Moderate-risk | | | | | |
| | Training Cohort | 92.6% | 76.4% | 59.7% | 47.9% |
| | Test Cohort | 67.8% | 56.7% | 33.1% | n/a |
| High-risk | | | | | |
| | Training Cohort | 81.1% | 54.4% | 24.9% | 15.6% |
| | Test Cohort | 62.1% | 34.1% | 17.1% | 8.5% |
| Very-high-risk | | | | | |
| | Training Cohort | 59.9% | 24.3% | 12.2% | 0% |
| | Test Cohort | 16.7% | 11.1% | 0% | 0% |
| Progression-free survival | | | | | |
| Overall by cohort | | | | | |
| | Training Cohort | 47.9% | 32.8% | 22.8% | 20.8% |
| | Test Cohort | 37.9% | 19.6% | 9.5% | 9.5% |
| By risk group | | | | | |
| Low-risk | | | | | |
| | Training Cohort | 71.4% | 71.4% | 65.5% | 65.5% |
| | Test Cohort | 73.7% | 46.3% | 29.8% | 29.8% |
| Moderate-risk | | | | | |
| | Training Cohort | 65.9% | 43.0% | 31.2% | 25.0% |
| | Test Cohort | 23.9% | 19.1% | 9.6% | n/a |
| High-risk | | | | | |
| | Training Cohort | 43.0% | 27.0% | 9.8% | 9.8% |
| | Test Cohort | 41.3% | 15.0% | 3.8% | n/a |
| Very-high-risk | | | | | |
| | Training Cohort | 15.7% | 0% | 0% | 0% |
| | Test Cohort | 11.1% | 0% | 0% | 0% |

¹Cells were marked as n/a whenever all of the patients were censored for the given interval.

Clinical model: Among the 16 clinical covariates from Table 1 that were considered for the clinical model, four clinical features (serum albumin, number of metastatic sites, previous lines of therapy and neutrophils counts) were significantly associated with OS in univariable analysis utilizing the training cohort. The final parsimonious clinical model included two clinical features: serum albumin (hazard ratio [HR]=0.33; 95% Cl:0.20-0.52) and number of metastatic sites (HR=2.14; 95% Cl: 1.48-3.11).

Radiomics model: Among the original 213 intratumoral and peritumoral radiomic features, 67 features were found to be stable and reproducible (FIG. 2A). Eight of the 67 features were removed because they were correlated with tumor volume. Univariable analysis identified eleven features significantly associated with OS and eight of the nine features were dropped because they were correlated within each other (FIG. 2B). Among the two remaining features (gray level co-occurrence matrix [GLCM] inverse difference and peritumoral quartile coefficient), stepwise backward elimination approach identified GLCM inverse difference as the most informative radiomic feature (HR=1.41; 95% Cl: 1.19-1.67, p<0.001).

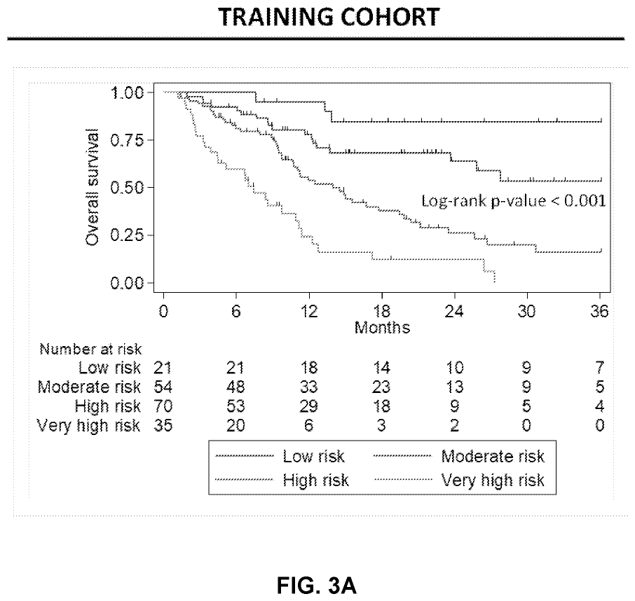

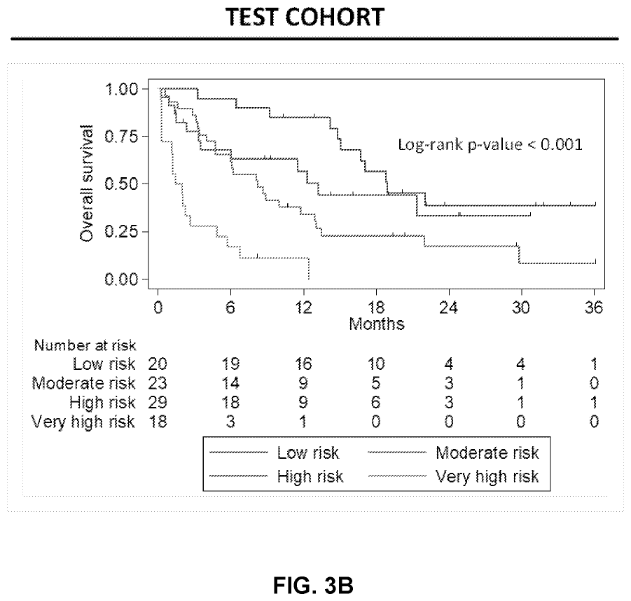

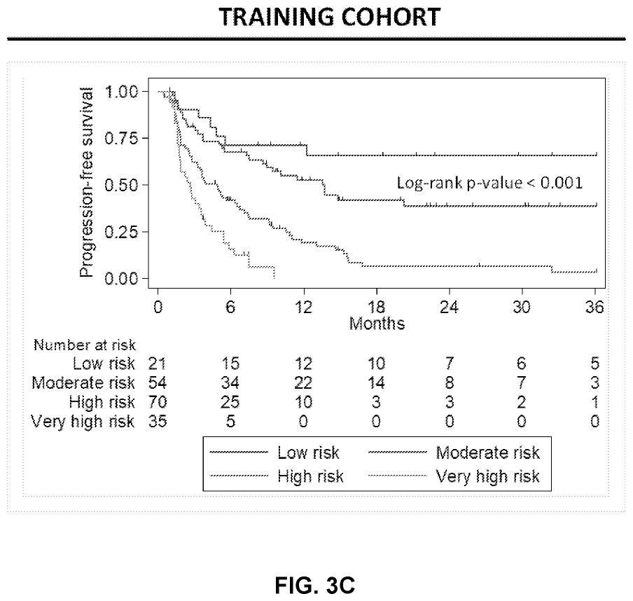

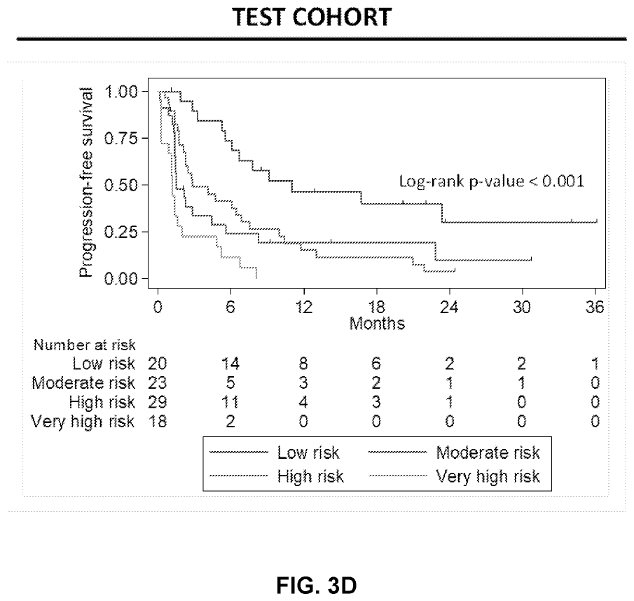

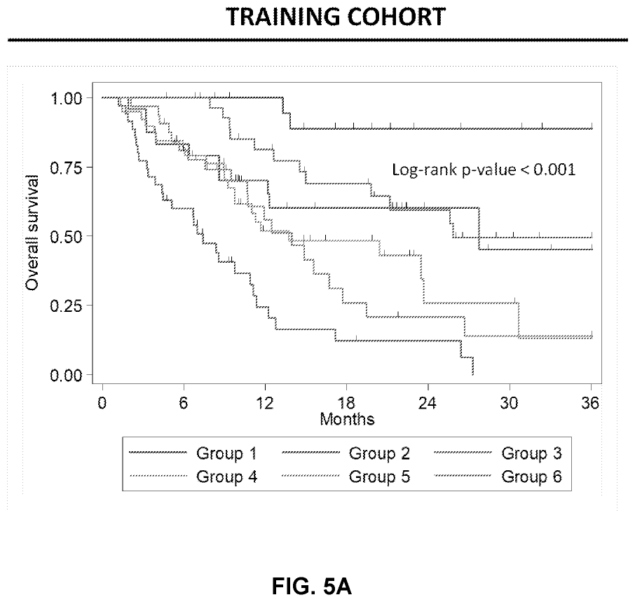

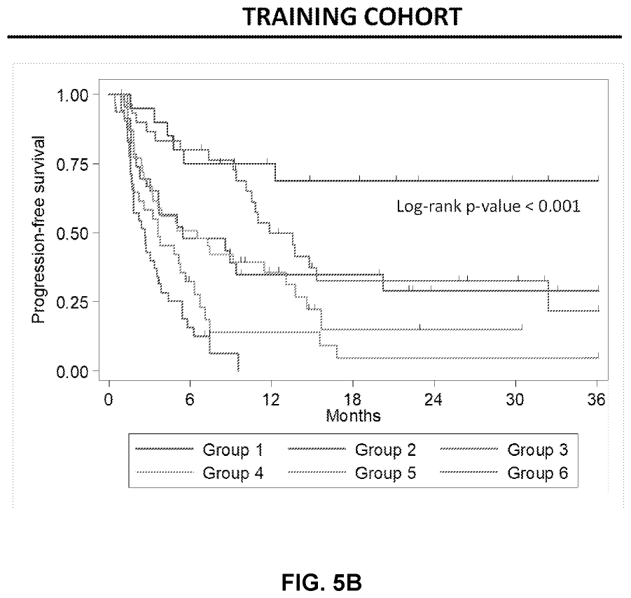

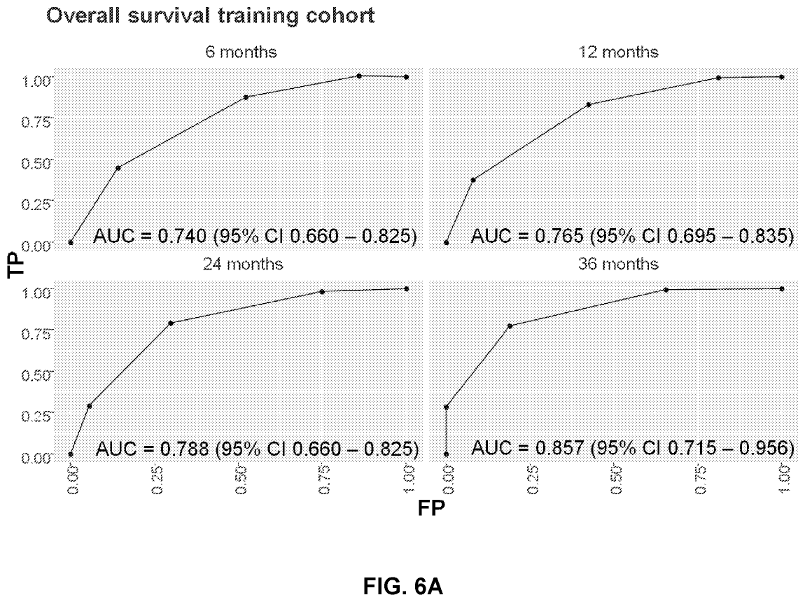

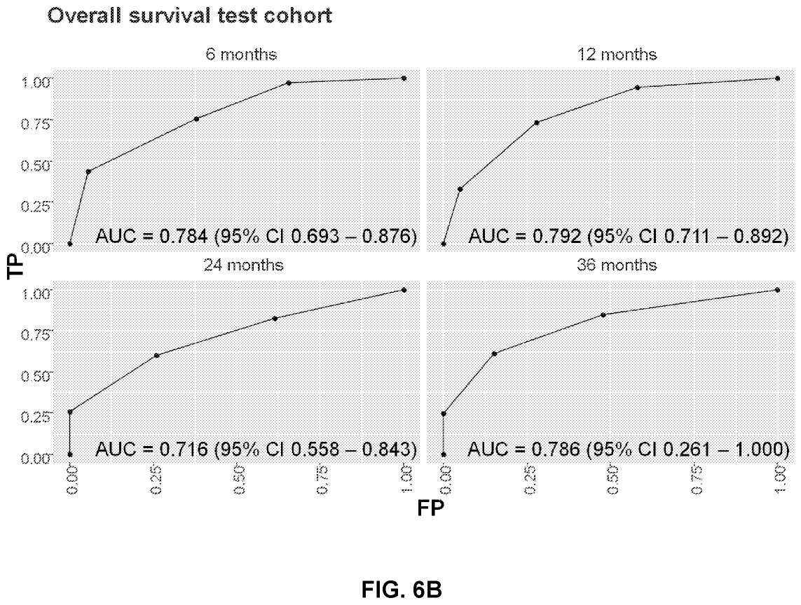

CART analysis: Based on the two most informative clinical covariates and most informative radiomic feature, CART analysis have found novel cut-off points (FIG. 2C) and classified patients in the training cohort into six risk groups (FIG. 5) which were further collapsed into four risk groups based on OS: low-risk, moderate-risk, high-risk, and very high-risk (FIG. 3). Similar findings were observed for PFS. The risk groups identified in the training cohort were also extracted in the test cohort (Table 2 and FIG. 3) where the time-dependent AUCs were found to be similar for both cohorts for OS (FIG. 6). Specifically, for 6 months OS, our model achieved an AUC of 0.784 (95% Cl: 0.693-0.876) and for 24 months the AUC was 0.716 (95% Cl: 0.558-0.843) for the test cohort.

Multivariable analysis: A multivariable Cox regression analysis was conducted adjusting for clinical covariates that were significantly different between the training cohort and test cohort (Table 1). The HRs were adjusted for ECOG, lymphocyte counts and neutrophils to lymphocytes ratio (Table 3) and the high-risk (test cohort HR=3.33; 95% C 1.57-7.05) and very high-risk (test cohort HR=5.35; 95% Cl 2.14-13.36) groups were still found to be associated with significantly worse outcomes compared to the low-risk group (HR=1.00). The results were consistent when the data were analyzed for PFS. Utilizing the validation cohort, the very-high risk group had significantly worse outcomes (HR=8.06; 95% C 1.78-36.44) compared to the low-risk group.

Clinical covariates were compared across the four CART risk groups (Table 4) and previous lines of therapy, ECOG, white blood cell counts, neutrophils and NLR were found to be significantly different. Multivariable Cox regression was performed adjusting for these potential confounders but did not appreciably alter the HRs for risk groups.

TABLE 3
Univariable and multivariable Cox regression analysis for overall
survival and progression-free survival for the training and test cohorts.

| Training cohort (N = 180) | | | Test cohort (N = 90) | | |
|---|---|---|---|---|---|
| Univar- | Multi- | Multi- | Univar- | Multi- | Multi- |
| iable | variable | variable | iable | variable | variable |
| Model¹ | Model² | Model³ | Model¹ | Model² | Model³ |
| HR | HR | HR | HR | HR | HR |
| (95% | (95% | (95% | (95% | (95% | (95% |
| CI) | CI) | CI) | CI) | CI) | CI) |

Overall survival

| | | | | | |
|---|---|---|---|---|---|
| Risk group | | | | | |
| Low-risk | 1.00 | 1.00 | 1.00 | 1.00 | 1.00 |
| | (Refer- | (Refer- | (Refer- | (Refer- | (Refer- |
| | ence) | ence) | ence) | ence) | ence) |
| Moderate- | 3.79 | 3.08 | 3.56 | 1.70 | 1.51 |
| risk | (1.13- | (0.89- | (1.02- | (0.75- | (0.66- |
| | 12.68) | 10.66) | 12.48) | 3.87) | 3.51) |
| High- | 8.02 | 7.87 | 6.98 | 2.73 | 3.33 |
| risk | (2.47- | (2.38- | (2.10- | (1.33- | (1.57- |
| | 26.09) | 25.97) | 23.18) | 5.63) | 7.05) |
| Very- | 19.32 | 17.33 | 17.24 | 10.52 | 5.35 |
| high- | (5.80- | (5.11- | (5.09- | (4.58- | (2.14- |
| risk | 64.32) | 58.72) | 58.36) | 24.17) | 13.36) |
| ECOG | — | 1.22 | 1.20 | — | 2.63 |
| | | (0.70- | (0.69- | | (1.47- |
| | | 2.11) | 2.07) | | 4.68) |
| Pr. | — | — | 1.36 | — | — |
| treatment | | | (1.01- | | |
| | | | 1.81) | | |
| Lympho | — | 1.04 | — | — | 0.73 |
| cytes | | (0.74- | | | (0.45- |
| | | 1.46) | | | 1.17) |
| WBC | — | — | 0.98 | — | — |
| | | | (0.88- | | |
| | | | 1.09) | | |
| Neutro- | — | — | 1.10 | — | — |
| phils | | | (0.89- | | |
| | | | 1.34) | | |
| NLR | — | 1.01 | 0.98 | — | 1.05 |
| | | (0.97- | (0.92- | | (1.02- |
| | | 1.06) | 1.05) | | 1.08) |

Progression-free survival

| | | | | | |
|---|---|---|---|---|---|
| Risk group | | | | | |
| Low- | 1.00 | 1.00 | 1.00 | 1.00 | 1.00 |
| risk | (Refer- | (Refer- | (Refer- | (Refer- | (Refer- |
| | ence) | ence) | ence) | ence) | ence) |
| Moderate- | 2.02 | 2.05 | 2.36 | 2.96 | 2.80 |
| risk | (0.89- | (0.88- | (1.00- | (1.43- | (1.34- |
| | 4.64) | 4.76) | 5.58) | 6.14) | 5.85) |
| High- | 5.15 | 5.55 | 4.89 | 2.58 | 3.05 |
| risk | (2.33- | (2.46- | (2.15- | (1.29- | (1.50- |
| | 11.36) | 12.49) | 11.14) | 5.14) | 6.18) |
| Very- | 9.62 | 9.03 | 8.79 | 7.13 | 3.95 |
| high- | (4.12- | (3.77- | (3.66- | (3.31- | (1.56- |
| risk | 22.44) | 21.63) | 21.11) | 15.35) | 8.54) |
| ECOG | — | 1.09 | 1.05 | — | 2.33 |
| | | (0.68- | (0.66- | | (1.35- |
| | | 1.74) | 1.68) | | 4.03) |
| Prv | — | — | 1.32 | | — |
| treatment | | | (1.04 - | | |
| | | | 1.67) | | |
| Lympho | — | 0.83 | — | — | 0.88 |
| cytes | | (0.63- | | | (0.59- |
| | | 1.09) | | | 1.33) |
| WBC | — | — | 1.00 | — | — |
| | | | (0.90- | | |
| | | | 1.11) | | |
| Neutro- | — | — | 1.04 | — | — |
| phils | | | (0.86- | | |
| | | | 1.26) | | |
| NLR | — | 1.04 | 1.01 | — | 1.05 |
| | | (0.99- | (0.95- | | (1.02- |
| | | 1.09) | 1.07) | | 1.08) |

Abbreviations:
SD = standard deviation;
HR = hazard ratio;
CI = confidence interval;
PFS = progression-free survival;
NLR = neutrophils to lymphocytes ratio;
WBC = white blood cell;
Pr. treatment = previous lines of treatments at current diagnosis
Bold values are statistically significant.
¹The main effects for each risk group with the low risk group as the referent category.
²These models included the clinical covariates that were found to be significant different between the training and test cohorts (Table 1) and the risk groups using the low risk group as the referent category.
³These models included the clinical covariates that were found to be significant different between the CART risk groups (Sup Table 2).

TABLE 4
Patient characteristics by CART risk groups for the training cohort.

| | | | | | |
|---|---|---|---|---|---|
| | | | | Very- | |
| | | Moderate | High | high | P- |
| Characteristic | Low risk | risk | risk | risk | Value |

Age at diagnosis, N (%)

| | | | | | |
|---|---|---|---|---|---|
| Dichotomized | | | | | |
| <65 | 9 (42.9) | 22 (40.7) | 26 | 11 | |
| | | | (37.1) | (31.4 ) | |
| ≥65 | 12 (57.1) | 32 (59.3) | 44 | 24 | 0.798 |
| | | | (62.9) | (68.6) | |

Sex, N (%)

| | | | | | |
|---|---|---|---|---|---|
| Female | 8 (38.1) | 31 (57.4) | 40 | 16 | |
| | | | (57.1) | (45.7) | |
| Male | 13 (61.9) | 23 (42.6) | 30 | 19 | 0.323 |
| | | | (42.9) | (54.3) | |

Smoking status¹

| | | | | | |
|---|---|---|---|---|---|
| Never smoker | 4 (19.1) | 10 (18.9) | 12 | 4 | |
| | | | (17.9) | (11.4) | |
| Ever smoker | 17 (80.9) | 43 (81.1) | 55 | 31 | 0.809 |
| | | | (82.1) | (88.6) | |

Stage, N (%)

| | | | | | |
|---|---|---|---|---|---|
| III | 2 (9.5) | 0 (0) | 3 | 1 | |
| | | | (4.3) | (2.9) | |
| IV | 19 (90.5) | 54 (100) | 67 | 34 | 0.138 |
| | | | (95.7) | (97.1) | |

Histology, N (%)

| | | | | | |
|---|---|---|---|---|---|
| Adenocarcinoma/ | 17 (81.0) | 43 (79.6) | 53 | 24 | |
| others | | | (75.7) | (68.6) | |
| Squamous cell | 4 (19.0) | 11 (20.4) | 17 | 11 | 0.636 |
| carcinoma | | | (24.3) | (31.4) | |

Checkpoint inhibitors, N (%)

| | | | | | |
|---|---|---|---|---|---|
| Anti PD-L1 | 4 (19.1) | 11 (20.37) | 24 | 9 | |
| | | | (34.3) | (25.7) | |
| Anti PD-1 | 7 (33.3) | 16 (29.6) | 25 | 9 | |
| | | | (35.7) | (25.7 ) | |
| Doublet | 10 (47.6) | 27 (50.0) | 21 | 17 | 0.285 |
| | | | (30.0) | (48.6) | |

ECOG performance status, N (%)

| | | | | | |
|---|---|---|---|---|---|
| 0 | 10 (47.6) | 10 (18.5) | 15 | 4 | |
| | | | (21.4) | (11.4) | |
| 1 | 11 (52.4) | 44 (81.5) | 55 | 31 | 0.021 |
| | | | (78.6 ) | (88.6) | |

Previous lines of therapy on current diagnosis

| | | | | | |
|---|---|---|---|---|---|
| None | 10 (47.6) | 33 (61.1) | 10 | 17 | |
| | | | (14.3) | (48.6) | |
| 1 | 4 (19.1) | 13 (24.1) | 24 | 7 | |
| | | | (34.3) | (20.0) | |
| ≥2 | 7 (33.3) | 8(14.8) | 36 | 11 | <0.001 |
| | | | (51.4 ) | (31.4) | |

Number of metastatic sites

| | | | | | |
|---|---|---|---|---|---|
| 1 | 21 (100) | 14 (25.9) | 47 | 0 (0) | |
| | | | (67.1) | | |
| ≥2 | 0 (0) | 40 (74.1) | 23 | 35 | <0.001 |
| | | | (32.9) | (100) | |

EGFR mutational status¹

| | | | | | |
|---|---|---|---|---|---|
| Not Detected | 14 (77.8) | 36 (87.8) | 37 | 20 | |
| | | | (75.5) | (83.3) | |
| Detected | 4 (22.2) | 5 (12.2) | 12 | 4 | 0.495 |
| | | | (24.5) | (16.7) | |

KRAS mutational status¹

| | | | | | |
|---|---|---|---|---|---|
| Not Detected | 7 (58.3) | 17 (60.7) | 26 | 11 | |
| | | | (70.3) | (84.6 ) | |
| Detected | 5 (41.7) | 11 (39.3) | 11 | 2 | 0.401 |
| | | | (29.7) | (15.4) | |

Hematology, median, (95% CI)

| | | | | | |
|---|---|---|---|---|---|
| Serum albumin, | 4.0 (3.8- | 4.1 (4.1- | 4.0 | 3.6 | <0.001 |
| (g/dL) | 4.2) | 4.2 | (3.9- | (3.5- | |
| | | | 4.1) | 3.7) | |
| Lymphocytes, | 1.0 (0.8- | 1.2 (1.2- | 1.4 | 1.2 | 0.215 |
| (1e+9/L) | 1.4) | 1.4) | (1.3- | (0.8- | |
| | | | 1.5) | 1.6) | |
| WBC, (1e+9/L) | 6.8 (5.1- | 6.9 (6.4- | 6.9 | 8.3 | 0.023 |
| | | | (6.4- | (7.4- | |
| | | | 7.4) | 10.9) | |
| Neutrophils, | 4.8 (3.7- | 4.7 (4.1- | 4.4 | 6.1 | 0.007 |
| (1e+9/L) | 6.4) | 5.3) | (3.9- | (5.1- | |
| | | | 4.9) | 7.4) | |
| Ratio of: | 4.1 (2.7- | 3.4 (2.8- | 3.1 | 4.6 | 0.004 |
| Neutrophils/ | 5.7) | 4.0) | (2.8- | (3.8- | |
| Lymphocytes | | | 3.7) | 7.0) | |

Abbreviations:
CI = confidence interval;
NLR = neutrophils to lymphocytes ratio;
Bold P-values are statistically significant and P-values for continuous variables were calculated using Kruskall-Wallis test and Fisher's Exact Test of categorical variables.
¹P-values for Smoking status, EGFR mutational status and KRAS mutational status were calculated for patients without missing/inconclusive data.
Majority of the training (95.3%) and the test cohort (86.7%) were self-reported white race.
Majority of the training (97.0%) and the test cohort (88.9%) were self-reported non- Hispanic.

Radiogenomics analyses: For two-group analysis, GLCM inverse difference was dichotomized at the previously determined CART threshold (0.43), which was similar to the mean (0.47) and median (0.45) values in the radiogenomics dataset. Correlation and two-group analyses identified 123 significant probesets representing 91 unique genes that were associated with the GLCM inverse difference radiomic feature (Table 5).

TABLE 5

| | | Log2 Ratio | | Summary Score |
|---|---|---|---|---|
| | | (High GLCM- | Correlation | (ABS(Log Ratio* |
| Probeset | p. value | Low GLCM) | Coefficient | Correlation)) |
| merck- | 1.32E−05 | 1.37067634 | 0.43947875 | 0.60238312 |
| NM_001216_at | | | | |
| merck2- | 2.49E−05 | 1.25084427 | 0.43354816 | 0.54230123 |
| DQ892208_at | | | | |
| merck- | 0.00052201 | 0.62135466 | 0.40805752 | 0.25354844 |
| NM_138435_at | | | | |
| merck2- | 0.00060147 | −1.1481004 | −0.4001422 | 0.45940336 |
| BC052608_at | | | | |
| merck2- | 0.00012962 | −1.1618262 | −0.4005297 | 0.46534588 |
| BC065305_at | | | | |
| merck- | 0.0005668 | −0.5058407 | −0.4008055 | 0.20274371 |
| NM_001009567 | | | | |
| _s_at | | | | |
| merck- | 8.23E−05 | −1.2075022 | −0.4012643 | 0.48452759 |
| NM_153264_s_at | | | | |
| merck- | 5.61E−05 | −0.6658857 | −0.4018154 | 0.26756308 |
| AK092659_at | | | | |
| merck2- | 0.00014383 | −0.5814361 | −0.4020413 | 0.23376133 |
| AJ515553_at | | | | |
| merck- | 2.38E−05 | −0.9316574 | −0.4022998 | 0.37480556 |
| NM_004165_at | | | | |
| merck- | 8.26E−06 | −0.6381128 | −0.4024529 | 0.25681032 |
| ENST00000370 | | | | |
| 678_s_at | | | | |
| merck- | 3.15E−06 | −0.976778 | −0.4032688 | 0.39390412 |
| NM_001449_at | | | | |
| merck2- | 1.83E−05 | −0.9439359 | −0.403611 | 0.38098291 |
| BX648828_at | | | | |
| merck- | 0.00066878 | −0.6690615 | −0.4036409 | 0.27006056 |
| NM_198098_at | | | | |
| merck2- | 6.08E−05 | −0.7543111 | −0.4037108 | 0.30452359 |
| BC050635_at | | | | |
| merck- | 7.66E−05 | −0.5602973 | −0.4042033 | 0.22647403 |
| AF077048_a_at | | | | |
| merck- | 4.98E−05 | −0.9469657 | −0.4048032 | 0.38333476 |
| AK123737_s_at | | | | |
| merck- | 0.00036435 | −0.6189621 | −0.4051589 | 0.25077801 |
| AK091353_at | | | | |
| merck- | 0.00080436 | −1.2588081 | −0.4052868 | 0.51017831 |
| NM_015717_at | | | | |
| merck- | 0.00016906 | −0.8749401 | −0.4055916 | 0.35486836 |
| NM_000231_at | | | | |
| merck2- | 1.21E−05 | −1.111172 | −0.4056218 | 0.45071561 |
| NM_004657_at | | | | |
| merck- | 0.00083371 | −2.1461486 | −0.4057728 | 0.87084873 |
| NM_003018_s_at | | | | |
| merck- | 0.00018315 | −0.607782 | −0.4062836 | 0.24693187 |
| NM_001233_at | | | | |
| merck- | 3.50E−05 | −1.0964704 | −0.4070207 | 0.44628616 |
| NM_006774_at | | | | |
| merck- | 2.66E−05 | −1.1172571 | −0.4074716 | 0.45525048 |
| NM_003956_at | | | | |
| merck- | 7.04E−06 | −1.4655951 | −0.4075839 | 0.59735299 |
| NM_198392_at | | | | |
| merck- | 0.00010101 | −1.108105 | −0.4078806 | 0.4519745 |
| NM_030820_at | | | | |
| merck- | 7.55E−06 | −0.9547848 | −0.4085315 | 0.39005969 |
| ENST00000368 | | | | |
| 557_at | | | | |
| merck- | 3.10E−06 | −0.9939747 | −0.4087707 | 0.40630776 |
| NM_032609_s_at | | | | |
| merck2- | 1.54E−05 | −1.0651872 | −0.4093878 | 0.43607461 |
| BM555890_a_at | | | | |
| merck- | 0.00080257 | −0.5545075 | −0.4100112 | 0.22735427 |
| NM_000316_at | | | | |
| merck- | 4.38E−05 | −0.6930741 | −0.4101268 | 0.28424828 |
| AK090694_at | | | | |
| merck2- | 0.00053941 | −0.738028 | −0.4102172 | 0.30275177 |
| NM_005264_at | | | | |
| merck- | 0.00017431 | −1.1057963 | −0.4107187 | 0.4541712 |
| NM_031911_a_at | | | | |
| merck- | 0.0001976 | −0.55913 | −0.4110705 | 0.22984182 |
| NM_153206_s_at | | | | |
| merck2- | 0.00030567 | −0.5648992 | −0.4119314 | 0.23269972 |
| Z26653_at | | | | |
| merck- | 6.96E−06 | −0.5718779 | −0.4120454 | 0.23563961 |
| AF074993_at | | | | |
| merck- | 0.00021943 | −0.8893726 | −0.4124148 | 0.36679045 |
| AK055621_at | | | | |
| merck- | 5.72E−06 | −0.630941 | −0.4125022 | 0.26026454 |
| NM_001753_at | | | | |
| merck- | 9.38E−05 | −0.6125667 | −0.4126099 | 0.25275112 |
| NM_030964_s_at | | | | |
| merck- | 4.83E−06 | −0.9069971 | −0.4128392 | 0.37444399 |
| AK093713_at | | | | |
| merck- | 1.77E−06 | −0.9420997 | −0.4165888 | 0.39246813 |
| NM_001035_a_at | | | | |
| merck2- | 1.12E−05 | −1.0456119 | −0.4183306 | 0.43741149 |
| EB387139_a_at | | | | |
| merck- | 4.80E−06 | −1.0748655 | −0.4187491 | 0.45009902 |
| NM_014917_at | | | | |
| merck2- | 1.84E−05 | −1.1474172 | −0.4187704 | 0.48050434 |
| NM_006774_at | | | | |
| merck- | 1.33E−05 | −0.5553457 | −0.4205481 | 0.23354959 |
| HSS00001975_s_at | | | | |
| merck- | 4.87E−05 | −0.7951839 | −0.4206972 | 0.33453168 |
| NM_002404_at | | | | |
| merck- | 6.39E−07 | −1.2567235 | −0.4209729 | 0.52904651 |
| NM_003206_a_at | | | | |
| merck- | 0.00075959 | −0.8967222 | −0.4211654 | 0.37766837 |
| NM_000139_at | | | | |
| merck- | 4.34E−07 | −0.8273946 | −0.4212195 | 0.34851477 |
| NM_022062_s_at | | | | |
| merck- | 1.71E−06 | −0.8234094 | −0.4212819 | 0.34688749 |
| NM_003189_at | | | | |
| merck- | 0.00011288 | −1.5829484 | −0.4213585 | 0.66698874 |
| AK057197_a_at | | | | |
| merck- | 0.00022796 | −0.5869363 | −0.4224352 | 0.24794256 |
| NM_000426_at | | | | |
| merck2- | 0.00011735 | −0.5547635 | −0.4224973 | 0.2343861 |
| AL832100_at | | | | |
| merck- | 0.00014673 | −1.3447094 | −0.4226301 | 0.56831465 |
| AY102069_at | | | | |
| merck- | 7.60E−05 | −0.5876287 | −0.4232791 | 0.24873095 |
| BC039203_at | | | | |
| merck2- | 5.42E−05 | −0.9422332 | −0.4234448 | 0.39898376 |
| BC062365_at | | | | |
| merck- | 7.97E−06 | −0.7125604 | −0.4237712 | 0.30196255 |
| NM_212464_s_at | | | | |
| merck- | 0.00038121 | −1.1753922 | −0.4239817 | 0.49834479 |
| BX648964_at | | | | |
| merck- | 0.00029845 | −0.6982506 | −0.4251395 | 0.29685389 |
| NM_152606_at | | | | |
| merck- | 1.88E−05 | −0.8915149 | −0.4259969 | 0.37978263 |
| AK123264_at | | | | |
| merck- | 2.76E−06 | −1.5585154 | −0.4261072 | 0.6640946 |
| BU681386_at | | | | |
| merck2- | 1.53E−05 | −0.7174608 | −0.4266646 | 0.30611512 |
| CB240565_at | | | | |
| merck- | 6.67E−06 | −0.943362 | −0.4267582 | 0.40258747 |
| NM_174934_at | | | | |
| merck- | 3.43E−06 | −0.7710433 | −0.4274638 | 0.32959308 |
| NM_002084_at | | | | |
| merck- | 7.56E−06 | −0.8878424 | −0.427639 | 0.37967605 |
| NM_018488_at | | | | |
| merck- | 7.58E−06 | −1.085831 | −0.4280223 | 0.46475989 |
| AL133118_at | | | | |
| merck- | 6.77E−05 | −0.9711127 | −0.4283673 | 0.41599289 |
| AK074308_at | | | | |
| merck- | 0.00023785 | −0.5474348 | −0.4285155 | 0.23458427 |
| NM_024315_at | | | | |
| merck- | 1.50E−05 | −1.4811293 | −0.4290331 | 0.6354535 |
| NM_153267_at | | | | |
| merck2- | 0.00026533 | −0.8043905 | −0.4297559 | 0.34569156 |
| BCO21053_at | | | | |
| merck- | 5.78E−05 | −0.9430434 | −0.4306488 | 0.4061205 |
| NM_021902_s_at | | | | |
| merck- | 9.05E−06 | −1.0633438 | −0.4313071 | 0.4586277 |
| BX106890_a_at | | | | |
| merck2- | 0.00010039 | −1.0780269 | −0.4316853 | 0.46536838 |
| NG_001111_s_at | | | | |
| merck2- | 5.32E−06 | −1.1357841 | −0.431809 | 0.49044174 |
| AI478811_at | | | | |
| merck- | 1.96E−06 | −1.0271732 | −0.4322544 | 0.44400011 |
| AK057923_at | | | | |
| merck- | 0.00068792 | −0.8730285 | −0.4323373 | 0.37744281 |
| NM_001870_at | | | | |
| merck- | 1.24E−05 | −1.2699382 | −0.4330307 | 0.54992223 |
| AL834346_at | | | | |
| merck- | 6.56E−05 | −1.5121984 | −0.4330324 | 0.65483085 |
| NM_002942_at | | | | |
| merck- | 8.33E−06 | −1.4709705 | −0.4337646 | 0.63805487 |
| NM_004962_at | | | | |
| merck- | 0.00051742 | −0.8326688 | −0.4340411 | 0.36141244 |
| NM_015215_at | | | | |
| merck- | 4.66E−06 | −0.8560943 | −0.434195 | 0.37171184 |
| AK058175_a_at | | | | |
| merck- | 7.79E−06 | −1.073685 | −0.434743 | 0.46677703 |
| NM_004484_at | | | | |
| merck- | 5.81E−05 | −0.7231179 | −0.4358663 | 0.31518271 |
| NM_152765_s_at | | | | |
| merck2- | 8.78E−06 | −1.1967526 | −0.4361066 | 0.52191172 |
| BC142620_at | | | | |
| merck2- | 0.00010797 | −1.5959891 | −0.4388975 | 0.70047567 |
| AV653866_at | | | | |
| merck2- | 4.83E−07 | −1.7018258 | −0.4400366 | 0.74886569 |
| NM_152547_at | | | | |
| merck2- | 1.83E−06 | −1.3437234 | −0.4401365 | 0.59142177 |
| AL831991_at | | | | |
| merck- | 1.48E−05 | −1.0183093 | −0.4426182 | 0.45072221 |
| NM_003638_a_at | | | | |
| merck- | 3.08E−05 | −0.8398621 | −0.4444064 | 0.37324008 |
| NM_058240_s_at | | | | |
| merck- | 7.18E−05 | −1.7077536 | −0.449986 | 0.76846517 |
| CD700286_s_at | | | | |
| merck- | 7.01E−07 | −1.6851321 | −0.4504326 | 0.75903841 |
| NM_152547_s_at | | | | |
| merck2- | 0.00011975 | −1.6731306 | −0.4510552 | 0.75467423 |
| X03350_at | | | | |
| merck- | 9.91E−05 | −0.955408 | −0.4515795 | 0.43144266 |
| ENST00000375 | | | | |
| 247_a_at | | | | |
| merck2- | 1.19E−05 | −0.8423573 | −0.4522541 | 0.38095958 |
| AK092282_at | | | | |
| merck- | 2.24E−05 | −1.0101551 | −0.4524334 | 0.4570279 |
| ENST00000378 | | | | |
| 076_at | | | | |
| merck- | 5.68E−08 | −1.0817427 | −0.4527226 | 0.48972935 |
| NM_016438_at | | | | |
| merck2- | 0.0001087 | −1.0857598 | −0.4532487 | 0.49211919 |
| NM_000500_at | | | | |
| merck2- | 2.33E−05 | −0.9985536 | −0.4566317 | 0.45597125 |
| BC047725_at | | | | |
| merck2- | 3.02E−05 | −1.521755 | −0.4575645 | 0.69630103 |
| ABV60894_at | | | | |
| merck- | 2.09E−05 | −0.7582378 | −0.4577101 | 0.3470531 |
| NM_001452_at | | | | |
| merck2- | 8.37E−05 | −0.9641112 | −0.4582927 | 0.44184508 |
| NM_014485_at | | | | |
| merck- | 1.16E−05 | −1.1242538 | −0.4588571 | 0.5158718 |
| NM_018334_at | | | | |
| merck- | 6.61E−06 | −1.2009199 | −0.4613012 | 0.5539858 |
| ENST00000298 | | | | |
| 441_a_at | | | | |
| merck2- | 4.04E−05 | −1.005138 | −0.4633231 | 0.46570367 |
| NM_019105_at | | | | |
| merck- | 5.18E−05 | −1.8033321 | −0.4652628 | 0.83902344 |
| BX647469_a_at | | | | |
| merck- | 3.02E−06 | −1.6138471 | −0.468823 | 0.75660872 |
| NM_018286_at | | | | |
| merck2- | 0.00043611 | −0.6992877 | −0.4693476 | 0.32820898 |
| AI620331_at | | | | |
| merck- | 2.99E−05 | −1.3453105 | −0.4715787 | 0.63441981 |
| NM_007037_at | | | | |
| merck- | 6.62E−05 | −0.9880504 | −0.4731356 | 0.46748182 |
| BC020734_a_at | | | | |
| merck2- | 2.38E−06 | −1.736491 | −0.4748902 | 0.82464263 |
| AK095175_at | | | | |
| merck2- | 3.54E−05 | −0.8468696 | −0.4763596 | 0.40341445 |
| BC057807_at | | | | |
| merck2- | 7.29E−07 | −1.2394889 | −0.4792794 | 0.59406143 |
| AK027375_at | | | | |
| merck- | 3.63E−05 | −1.3210887 | −0.4809428 | 0.63536813 |
| NM_002001_at | | | | |
| merck- | 2.93E−07 | −1.3925361 | −0.4812479 | 0.67015506 |
| NM_030569_at | | | | |
| merck2- | 1.51E−07 | −0.8573412 | −0.4862095 | 0.41684749 |
| BI597924_at | | | | |
| merck2- | 6.37E−07 | −1.2664987 | −0.4912937 | 0.62222284 |
| NM_020482_at | | | | |
| merck- | 0.0007466 | −0.8262373 | −0.4920278 | 0.40653178 |
| NM_014279_at | | | | |
| merck- | 3.07E−05 | −1.164078 | −0.4992386 | 0.58115263 |
| NM_001007544_at | | | | |
| merck- | 9.59E−08 | −0.918037 | −0.4998211 | 0.45885432 |
| NM_014059_s_at | | | | |
| merck- | 8.79E−06 | −1.053207 | −0.5053559 | 0.53224433 |
| NM_001765_at | | | | |
| merck- | 3.30E−07 | −2.1858567 | −0.5065212 | 1.10718285 |
| NM_004469_at | | | | |
| merck2- | 8.20E−06 | −1.0896287 | −0.5083878 | 0.55395396 |
| AK226024_at | | | | |

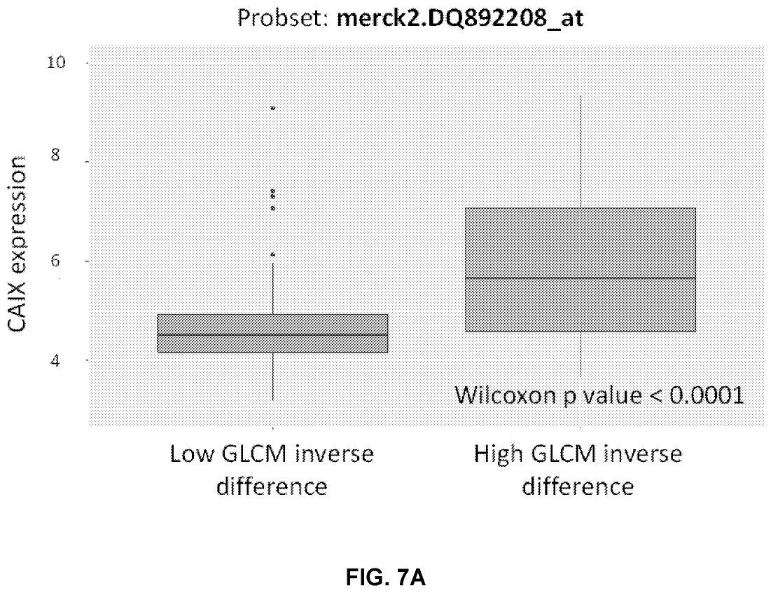

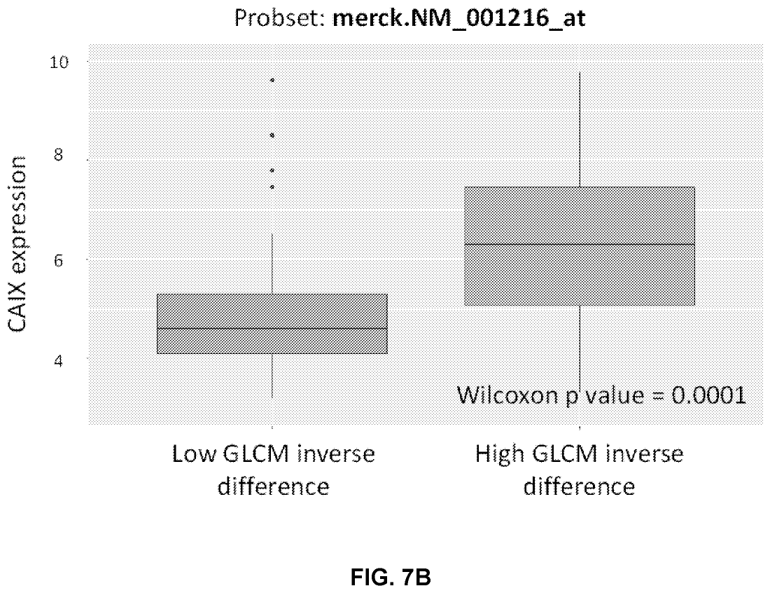

Pathway analysis indicated no significant enrichment (FOR<0.05). Gene Ontology Biological Process enrichment of the gene set identified terms including regulation of cardiac conduction, sodium ion export across plasma membrane and membrane depolarization during action potential (Table 6). Interestingly, only three probesets (representing two genes) were positively associated with GLCM inverse difference: carbonic anhydrase IX (CAIX) and Family With Sequence Similarity 83 Member F (FAM83F). GLCM inverse difference was positively associated with CAIX expression based on two different probesets (FIG. 7). Median CAIX expression was lower for patients with low GLCM inverse difference (<0.43) (merck2-DQ892208_at: 4.61 (95% Cl: 4.38-5.00); merck-NM_001216_at: 4.48 (95% Cl: 4.24-4.62)) vs. high GLCM inverse difference (≥0.43) (merck2-DQ892208_at: 6.32 (95% Cl: 5.50-6.86); merck-NM_001216_at: 5.66 (95% Cl: 5.11-6.39)).

TABLE 6
Enrichment Analysis Report

| Enrichment by GO Processes | | | | IORadiomics TwoGroup Correlation Intersection | | | |
|---|---|---|---|---|---|---|---|
| | | Min | Min | p- | | In | Network Objects from |
| Processes | Total | (pValue) | FDR | value | FDR | Data | Active Data |
| regulation of | 89 | 3.040 | 7.675 | 3.040 | 7.675 | 7 | Phospholemman, ATP1A2, NCX, Ryanodine |
| cardiac | | E-08 | E-05 | E- | E-05 | | receptor 2, NCX3, ATP1alpha subunit, |
| conduction | | | | 08 | | | Caveolin-1 |
| sodium ion | 30 | 6.696 | 8.453 | 6.696 | 8.453 | 5 | ATP1A2, NCX, NCX3, ATP1alpha subunit, |
| export across | | E-08 | E-05 | E- | E-05 | | SCN7A |
| plasma | | | | 08 | | | |
| membrane | | | | | | | |
| heart process | 168 | 1.529 | 1.287 | 1.529 | 1.287 | 8 | Gamma-sarcoglycan, GPX, SCN4B, ATP1A2, |
| | | E-07 | E-04 | E- | E-04 | | NCX, Ryanodine receptor 2, ATP1alpha |
| | | | | 07 | | | subunit, Caveolin-1 |
| membrane | 46 | 6.159 | 3.625 | 6.159 | 3.625 | 5 | SCN4B, ATP1A2, NCX, ATP1alpha subunit, |
| depolarization | | E-07 | E-04 | E- | E-04 | | SCN7A |
| during | | | | 07 | | | |
| action | | | | | | | |
| potential | | | | | | | |
| export across | 48 | 7.651 | 3.625 | 7.651 | 3.625 | 5 | ATP1A2, NCX, NCX3, ATP1alpha subunit, |
| plasma | | E-07 | E-04 | E- | E-04 | | SCN7A |
| membrane | | | | 07 | | | |
| regulation of | 147 | 9.515 | 3.625 | 9.515 | 3.625 | 7 | Phospholemman, ATP1A2, NCX, Ryanodine |
| striated | | E-07 | E-04 | E- | E-04 | | receptor 2, NCX3, ATP1alpha subunit, |
| muscle | | | | 07 | | | Caveolin-1 |
| contraction | | | | | | | |
| heart | 150 | 1.090 | 3.625 | 1.090 | 3.625 | 7 | Gamma-sarcoglycan, GPX, SCN4B, ATP1A2, |
| contraction | | E-06 | E-04 | E- | E-04 | | NCX, Ryanodine receptor 2, ATP1alpha subunit |
| | | | | 06 | | | |
| regulation of | 52 | 1.148 | 3.625 | 1.148 | 3.625 | 5 | ATP1A2, NCX, Ryanodine receptor 2, |
| the force of | | E-06 | E-04 | E- | E-04 | | ATP1alpha subunit, Caveolin-1 |
| heart | | | | 06 | | | |
| contraction | | | | | | | |
| sodium ion | 54 | 1.390 | 3.899 | 1.390 | 3.899 | 5 | C7, ATP1A2, NCX, ATP1alpha subunit, SCN7A |
| homeostasis | | E-06 | E-04 | E- | E-04 | | |
| | | | | 06 | | | |
| regulation of | 510 | 1.925 | 4.447 | 1.925 | 4.447 | 11 | Phospholemman, SCN4B, ATP1A2, FHL1 |
| cation | | E-06 | E-04 | E- | E-04 | | (SLIM1), NCX, Ryanodine receptor 2, Calpain |
| transmembrane | | | | 06 | | | 3, RRAD, ATP1alpha subunit, Caveolin-1, Fc |
| transport | | | | | | | epsilon RI beta |
| regulation of | 89 | 3.040 | 7.675 | 3.040 | 7.675 | 7 | Phospholemman, ATP1A2, NCX, NCX3, |
| cardiac | | E-08 | E-05 | E- | E-05 | | Ryanodine receptor 2, ATP1alpha subunit, |
| conduction | | | | 08 | | | Caveolin-1 |
| sodium ion | 30 | 6.696 | 8.453 | 6.696 | 8.453 | 5 | ATP1A2, NCX, NCX3, ATP1alpha subunit, |
| export across | | E-08 | E-05 | E- | E-05 | | SCN7A |
| plasma | | | | 08 | | | |
| membrane | | | | | | | |
| heart process | 168 | 1.529 | 1.287 | 1.529 | 1.287 | 8 | Gamma-sarcoglycan, GPX, ATP1A2, SCN4B, |
| | | E-07 | E-04 | E- | E-04 | | NCX, Ryanodine receptor 2, ATP1alpha |
| | | | | 07 | | | subunit, Caveolin-1 |
| membrane | 46 | 6.159 | 3.625 | 6.159 | 3.625 | 5 | ATP1A2, SCN4B, NCX, ATP1alpha subunit, |
| depolarization | | E-07 | E-04 | E- | E-04 | | SCN7A |
| during | | | | 07 | | | |
| action | | | | | | | |
| potential | | | | | | | |
| export across | 48 | 7.651 | 3.625 | 7.651 | 3.625 | 5 | ATP1A2, NCX, NCX3, ATP1alpha subunit, |
| plasma | | E-07 | E-04 | E- | E-04 | | SCN7A |
| membrane | | | | 07 | | | |
| regulation of | 147 | 9.515 | 3.625 | 9.515 | 3.625 | 7 | Phospholemman, ATP1A2, NCX, NCX3, |
| striated | | E-07 | E-04 | E- | E-04 | | Ryanodine receptor 2, ATP1alpha subunit, |
| muscle | | | | 07 | | | Caveolin-1 |
| contraction | | | | | | | |
| heart | 150 | 1.090 | 3.625 | 1.090 | 3.625 | 7 | Gamma-sarcoglycan, GPX, ATP1A2, SCN4B, |
| contraction | | E-06 | E-04 | E- | E-04 | | NCX, Ryanodine receptor 2, ATP1alpha subunit |
| | | | | 06 | | | |
| regulation of | 52 | 1.148 | 3.625 | 1.148 | 3.625 | 5 | ATP1A2, NCX, Ryanodine receptor 2, |
| the force of | | E-06 | E-04 | E- | E-04 | | ATP1alpha subunit, Caveolin-1 |
| heart | | | | 06 | | | |
| contraction | | | | | | | |
| sodium ion | 54 | 1.390 | 3.899 | 1.390 | 3.899 | 5 | C7, ATP1A2, NCX, ATP1alpha subunit, SCN7A |
| homeostasis | | E-06 | E-04 | E- | E-04 | | |
| | | | | 06 | | | |
| regulation of | 510 | 1.925 | 4.447 | 1.925 | 4.447 | 11 | Phospholemman, ATP1A2, SCN4B, FHL1 |
| cation | | E-06 | E-04 | E- | E-04 | | (SLIM1), NCX, Ryanodine receptor 2, Calpain |
| transmembrane | | | | 06 | | | 3, RRAD, ATP1alpha subunit, Caveolin-1, Fc |
| transport | | | | | | | epsilon RI beta |

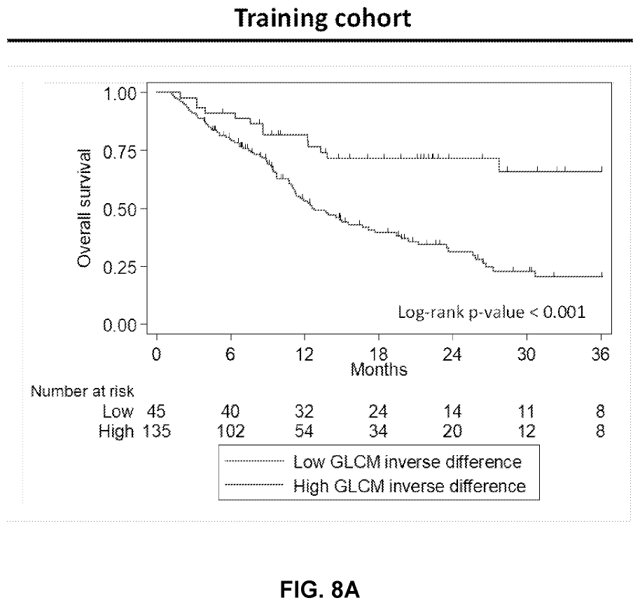

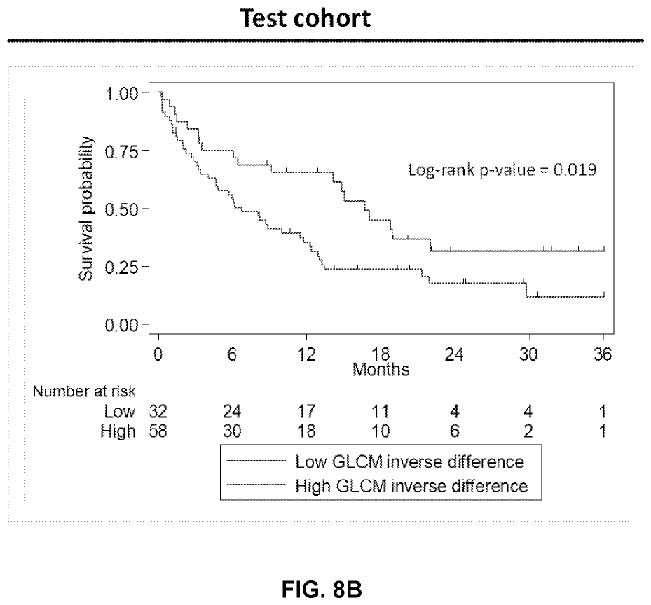

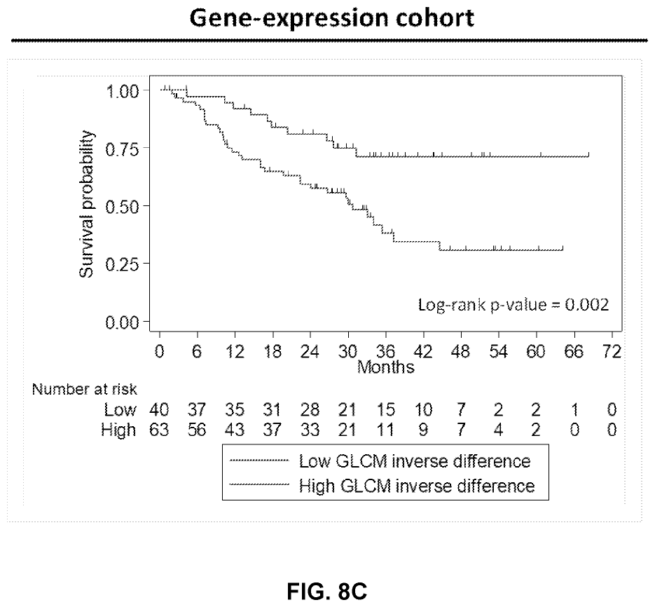

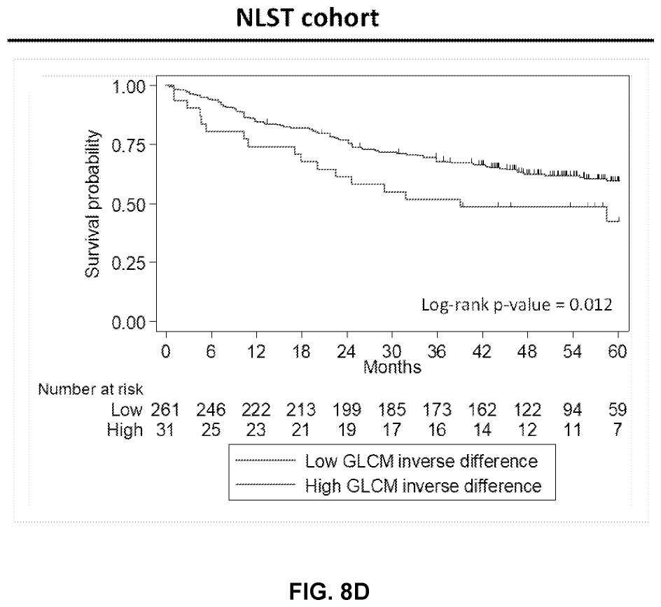

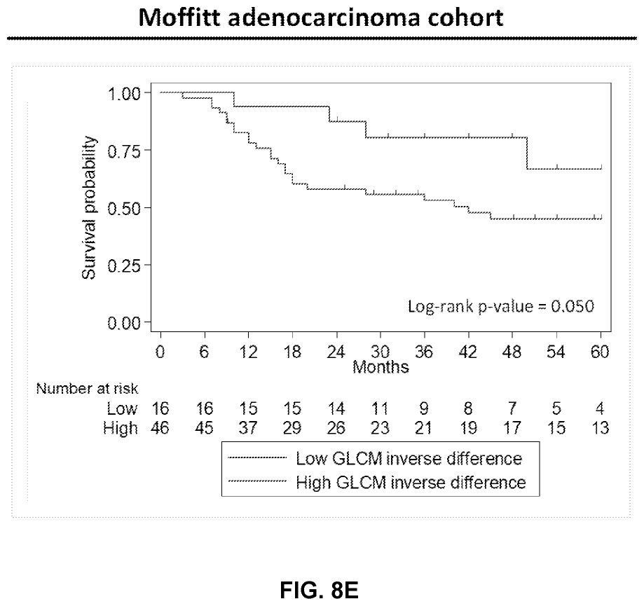

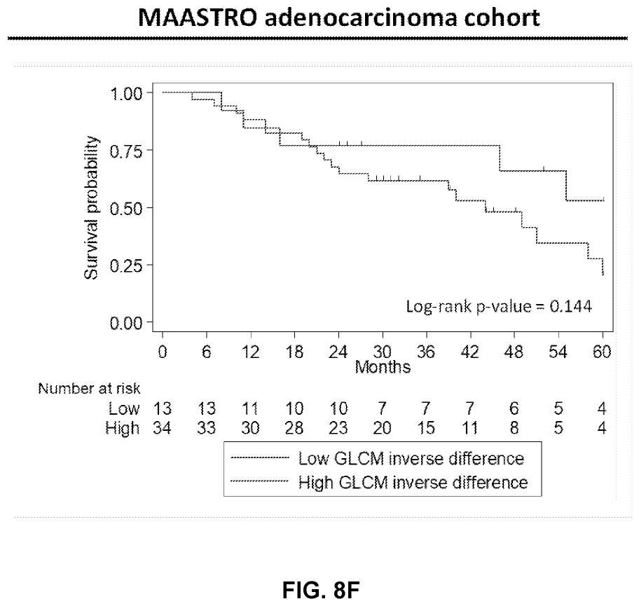

Prognostic validation datasets: GLCM inverse difference was significantly associated with OS in three out of the four independent NSCLC cohorts (FIG. 8) using previously found CART cut-point (0.43). Although the a priori cut-point for GLCM inverse difference was not significantly associated with OS in the Maastricht patient cohort, GLCM inverse difference as a continuous covariates was significantly associated with OS in a Cox regression model (HR=2.74; 95% C 1.04-7.24).

### DISCUSSION

Predictive biomarkers that identify lung cancer patients who will experience rapid and lethal outcomes is a critical unmet need as such, patients could avoid ineffective and expensive treatment. In this study, a rigorous radiomics pipeline was utilized to conduct a robust analysis to identify and successfully tested and validated a parsimonious clinical-radiomic model that was significantly associated with survival outcomes and stratified patients into four unique risk groups based on risk of patient death and risk of progression. The very high-risk group was associated with extremely poor OS and PFS in all the training test and independent validation cohorts (FIG. 3) which may suggest these patients should either avoid immunotherapy altogether or utilize upfront combination treatments that may yield a better response. The most informative radiomic feature, GLCM inverse difference, was positively associated with CAIX expression and further validation demonstrated that GLCM inverse difference was also associated with OS in four independent NSCLC cohorts.

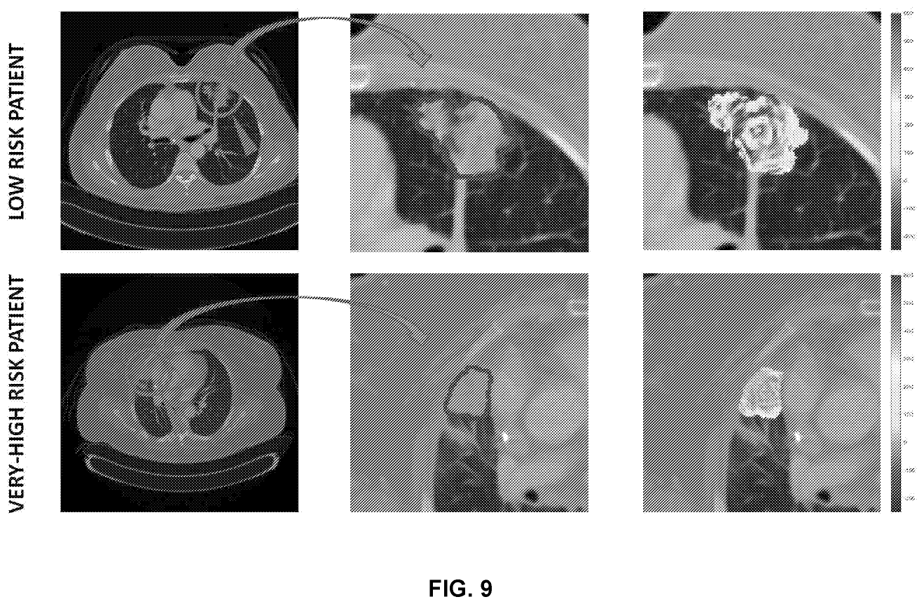

The four final risk groups found in this study were derived from one radiomic feature (GLCM inverse difference) and two clinical covariates (number of metastatic sites and serum albumin). Higher GLCM inverse difference was associated with poor outcomes in four other prognostic validation NSCLC cohorts suggesting a pan-radiomic feature. The GLCM inverse difference is an “avatar feature” that is correlated with nine other radiomic features (FIG. 2B). Dense, uniform lesions were less likely to respond to treatments as tumors with higher GLCM inverse difference were reflecting this phenotype (FIG. 9). Furthermore, analyses revealed that this avatar feature is associated tumor hypoxia since it was positively associated with CAIX expression which is an important pH regulatory enzyme that is upregulated in hypoxic tumors leading to an acidic tumor microenvironment (Traverso, A., et al. Int J Radiat Oncol Biol Phys (2018)) and associated with poor prognosis (Harris, A. L. Nat Rev Cancer 2:38-47 (2002); Chan, D. A. & Giaccia, A. J. Cancer Metastasis Rev 26:333-339 (2007)) including NSCLC (Ilie, M. et al. Lung Cancer 82:16-23 (2013); Pastorek, J. & Pastorekova, S. Semin Cancer Biol 31:52-64 (2015)). Tumor-hypoxia leads to advanced but dysfunctional vascularization and acquisition of epithelial-mesenchymal transition phenotype, resulting in cell mobility and metastasis and alters cancer cell metabolism and contributes to therapy resistance by inducing cell quiescence and immunosuppressive phenotype (Muz, B., et al. Hypoxia (Auckl) 3:83-92 (2015)). The most predictive clinical covariates in this study demonstrate the utility of standard-of-care clinical information to predictive treatment response. Higher number of metastatic sites increases disease burden and can result in mixed responses where one or more lesions may be responding while others are progressing and ultimately resulting in progressive disease. The other clinical covariate, serum albumin, has been shown to be associated with survival in NSCLC patients (Espinosa, E. et al. Lung Cancer 12:67-76 (1995); Miura, K. et al. Lung Cancer 111:88-95 (2017)) and is used in cancer prognostic scores including Royal Marsden Hospital prognostic score (Garrido-Laguna, I. et al. Cancer 118:1422-1428 (2012)) and MD Anderson risk score (Wheler, J. et al. Clin Cancer Res 18:2922-2929 (2012)). Lower serum albumin is an indicator of malnutrition, inflammation, and hepatic dysfunction which may lead to worse outcomes. The mechanism of serum albumin in related to immunotherapy response is not yet established yet.

Emerging evidence demonstrates the utility of radiomics as a non-invasive approach to quantify and predict lung cancer treatment response of tyrosine kinase inhibitors (Jia, T. Y. et al. Eur Radiol (2019); Aerts, H. J. W. L. et al. Sci Rep. 6:33860 (2016)), platinum-based chemotherapy (Khorrami, M. et al. Radiology: Artificial Intelligence 1 (2019)), neo-adjuvant chemo-radiation (Bibault, J. E. et al. Sci Rep. 8:12611 (2018); Coroller, T. P. et al. J Thorac Oncol. 12(3):467-476 (2017)), stereotactic body radiation therapy (Huynh, E. et al. Plos One 12 (2017); Mattonen, S. A. et al. Int J Radiat Oncol Biol Phys 94:1121-1128 (2016)), and immunotherapy (Tunali, I. et al. Lung Cancer 129:75-79 (2019); Sun, R. et al. Lancet Oncol 19:1180-1191 (2018); Trebeschi, S. et al. Ann Oncol (2019)). With respect to immunotherapy treatment response, pre-treatment clinical covariates and radiomic features predicted rapid disease progression phenotypes, including hyperprogression (AUROCs ranging 0.804-0.865) among 228 NSCLC patients treated with single agent or double agent immunotherapy (Tunali, I. et al. Lung Cancer 129:75-79 (2019)). A radiomic signature for 0d8 cells was developed that predicted clinical outcomes (AUC=0.67) among 135 patients spanning 15 different cancer types treated with anti-PD-1 or anti-PD-L1 NSCLC patients only represented 22% of their dataset (Sun, R. et al. Lancet Oncol 19:1180-1191 (2018)). A machine learning model that significantly discriminated progressive disease from stable and responsive disease (AUC=0.83) among 123 NSCLC patients treated with anti-PD1 immunotherapy was developed (Trebeschi, S. et al. Ann Oncol (2019)). The study presented here represents the single largest study population of NSCLC patients treated with immunotherapy.

This study is yields a high radiomic quality score (RQS=17) (Lambin, P. et al. Nat Rev Olin Oncol 14:749-762 (2017)) (Table 7), which is a stringent metric that quantifies the clinical relevance of a radiomic study.

TABLE 7
Radiomic quality score calculation

| Criteria | Point system | Study point |
|---|---|---|
| Image protocol quality—well-documented | +1 (if protocols are well- | 2 |
| image protocols (for example, contrast, slice | documented) +1 (if | |
| thickness, energy, etc.) and/or usage of public | public protocol is used) | |
| image protocols allow reproducibility/ | | |
| replicability | | |
| Multiple segmentations—possible actions are: | 1 | 1 |
| segmentation by different physicians/ | | |
| algorithms/software, perturbing segmentations | | |
| by (random) noise, segmentation at different | | |
| breathing cycles. | | |
| Analyze feature robustness to segmentation | | |
| variabilities | | |
| Phantom study on all scanners—detect | 1 | 0 |
| inter-scanner differences and vendor-dependent | | |
| features. Analyze feature robustness to these | | |
| sources of variability | | |
| Imaging at multiple time points—collect | 1 | 1 |
| images of individuals at additional time points. | | |
| Analyze feature robustness to temporal | | |
| variabilities (for example, organ movement, | | |
| organ expansion/shrinkage) | | |
| Feature reduction or adjustment for multiple | −3 (ft neither measure is | 3 |
| testing—decreases the risk of overfitting. | implemented) +3 (if | |
| Overfitting is inevitable if the number of | either measure is | |
| features exceeds the number of samples. | implemented) | |
| Consider feature robustness when selecting | | |
| features | | |
| Multivariable analysis with non radiomics | 1 | 1 |
| features (for example, EGFR mutation)—is | | |
| expected to provide a more holistic model. | | |
| Permits correlating/inferencing between | | |
| radiomics and non radiomics features | | |
| Detect and discuss biological correlates— | 1 | 1 |
| demonstration of phenotypic differences | | |
| (possibly associated with underlying | | |
| gene-protein expression patterns) deepens | | |
| understanding of radiomics and biology | | |
| Cut-off analyses—determine risk groups by | 1 | 1 |
| either the median, a previously published | | |
| cut-off or report a continuous risk variable. | | |
| Reduces the risk of reporting overly optimistic | | |
| results | | |
| Discrimination statistics—report discrimination | +1 (if a discrimination | 1 |
| statistics (for example, C-statistic, ROC curve, | statistic and its statistical | |
| AUC) and their statistical significance (for | significance are reported) | |
| example, p-values, confidence intervals). One | +1 (if a resampling | |
| can also apply resampling method (for | method technique is also | |
| example, bootstrapping, cross-validation) | applied) | |
| Calibration statistics—report calibration | +1 (if a calibration | 0 |
| statistics (for example, Calibration-in-the- | statistic and its statistical | |
| large/slope, calibration plots) and their | significance are reported), | |
| statistical significance (for example, P-values | +1 (if a resampling | |
| confidence intervals). One can also apply | method technique is also | |
| resampling method (for example, bootstrapping, | applied) | |
| cross-validation) | | |
| Prospective study registered in a trial | +7 (for prospective | 0 |
| database—provides the highest level of | validation of a radiomics | |
| evidence supporting the clinical validity and | signature in an | |
| usefulness of the radiomics biomarker | appropriate trial) | |
| Validation—the validation is performed without | −5 (if validation is | 3 |
| retraining and without adaptation of the cut-off | missing) +2 (if validation | |
| value, provides crucial information with regard | is based on a dataset | |
| to credible clinical performance | from the same institute) +3 | |
| | (if validation is based on a | |
| | dataset from another | |
| | institute) +4 (if validation | |
| | is based on two datasets | |
| | from two distinct institutes) | |
| | +4 (if the study validates | |
| | a previously published | |
| | signature) +5 (if | |
| | validation is based on | |
| | three or more datasets | |
| | from distinct institutes) | |
| Comparison to ‘gold standard’—assess the | 2 | 0 |
| extent to which the model agrees with/is | | |
| superior to the current ‘gold standard’ method | | |
| (for example, TNM-staging for survival | | |
| prediction). This comparison shows the added | | |
| value of radiomics | | |
| Potential clinical utility—report on the current | 2 | 2 |
| and potential application of the model in a | | |
| clinical setting (for example, decision curve | | |
| analysis) | | |
| Cost-effectiveness analysis—report on the | 1 | 0 |
| cost-effectiveness of the clinical application | | |
| (for example, QALYs generated) | | |
| Open science and data—make code and data | +1 (if scans are open | 1 |
| publicly available. Open science facilitates | source) +1 (if region of | |
| knowledge transfer and reproducibility of the | interest segmentations | |
| study | are open source) +1 (if | |
| | code is open source) +1 | |
| | (if radiomics features are | |
| | calculated on a set of | |
| | representative ROIs and | |
| | the calculated features | |
| | and representative ROls | |
| | are open source) | |
| | TOTAL SCORE | 17 |

In conclusion, using standard-of-care imaging and clinical covariates a new parsimonious model that predicts OS and PFS among NSCLC patients treated with immunotherapy was identified and validated. The potential clinical application of this work is that baseline radiomics and clinical covariates can identify patients that are unlikely to respond to immunotherapy.

### Example 2: Clinical-Radiomic Models Predict Overall Survival Among Non-Small Cell Lung Cancer Patients Treated with Immunotherapy

Checkpoint blockade immunotherapy provides improved long-term survival in a subset of advanced stage non-small cell lung cancer (NSCLC) patients. However, highly predictive biomarkers of immunotherapy response are an unmet clinical need; hence baseline (pre-treatment) clinical factors and radiomic features were utilized to identify risk models that predict survival outcomes among NSCLC patients treated with immunotherapy.

Methods

The NSCLC patients treated with immunotherapy were split into training (N=180) and test cohorts (N=90). Overall survival (OS) and progression-free survival (PFS) were the main endpoints. Among the most predictive and reproducible clinical and radiomic features, Classification and Regression Tree was used to stratify patients into risk-groups in the training cohort and validated in the test cohort. The biological underpinnings of the most informative radiomic feature were assessed using gene expression data from a radiogenomics dataset. Four independent NSCLC cohorts were utilized for further validation.

Results

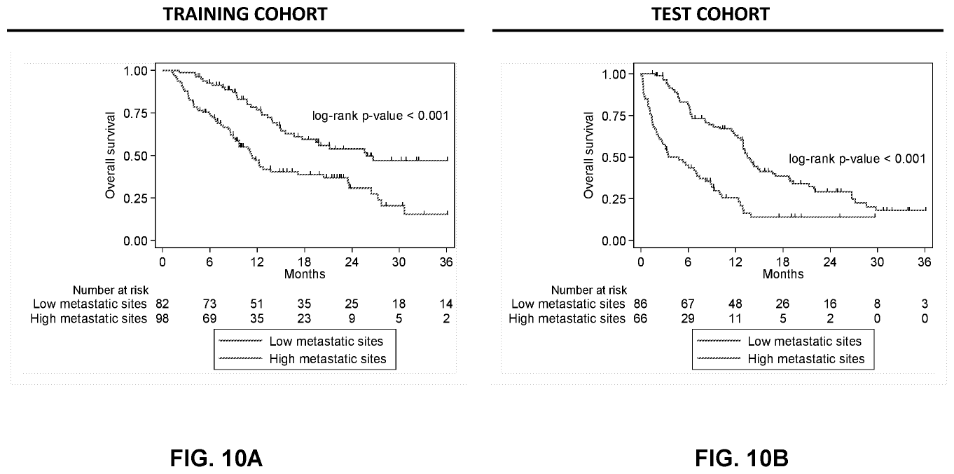

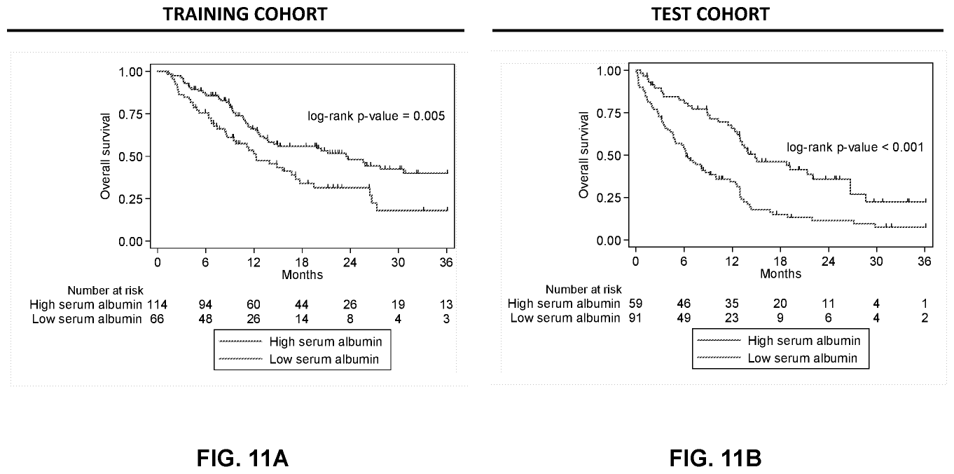

The analyses successfully validated a parsimonious model that was significantly associated with survival and stratified patients into risk groups: low-, moderate-, high-, and very-high risk (FIG. 10). In the test cohort, very-high risk group (N=18 [20%]) was associated with extremely poor OS (0% 3-year OS; hazard ratio [HR]=5.35, 95% confidence interval [Cl]: 2.14-13.36) compared to low-risk group (38.9% 3-year OS; HR=1.00). Similar findings were observed with PFS (0% vs. 29.8% 3-year PFS). The most informative radiomic feature, GLCM inverse difference, was positively associated with CAIX expression which is a marker of tumor hypoxia (Wilcoxon P-value=0.0001). GLCM inverse difference was also significantly associated with OS in four independent NSCLC cohorts.

### DISCUSSION

Disclosed herein are risk groups models that predict OS and PFS among NSCLC patients treated with immunotherapy. This model has important translational implications to identify a highly vulnerable subset of patients not likely to benefit from immunotherapy.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for predicting efficacy of immunotherapy in a subject with lung cancer, comprising
(a) receiving image data from contrast-enhanced thoracic computed tomography (CT) scans; and
(b) using a data processor to process the image data for gray level co-occurrence matrix (GLCM) inverse difference textures;
wherein high GLCM inverse difference indicates reduced efficacy of the immunotherapy in the subject.

2. The method of claim 1, further comprising determining the number of metastatic sites in the subject, wherein elevated metastatic sites indicates reduced efficacy of the immunotherapy in the subject.

3. The method of claim 1, further comprising assaying a blood sample from the subject for serum albumin, wherein a high GLCM inverse difference, decreased levels of serum albumin and higher number of metastatic sites indicates reduced efficacy of the immunotherapy in the subject.

4. A method for treating a subject with lung cancer, comprising
(c) receiving image data from contrast-enhanced thoracic computed tomography (CT) scans; and
(d) using a data processor to process the image data and detect low gray level co-occurrence matrix (GLCM) inverse difference texture feature indicative of less dense, and less uniform lesions;
(e) treating the subject with immunotherapy.

5. The method of claim 4, further comprising detecting no more than 1 metastatic sites in the subject.

6. The method of claim 4, wherein the immunotherapy comprises a checkpoint inhibitor.

7. The method of claim 6, wherein the checkpoint inhibitor comprises an anti-PD-1 antibody, anti-PD-L1 antibody, anti-CTLA-4 antibody, or a combination thereof.
